(19) United States
(12) Patent Application Publication
(10) Pub. No.: US 2021/0161416 A1
(43) Pub. Date: Jun. 3, 2021

(54) BIOMETRIC DATA MEASUREMENT SYSTEM AND INFORMATION PROCESSING DEVICE

(71) Applicant: OMRON HEALTHCARE Co., Ltd., Kyoto (JP)

(72) Inventors: Takanobu YAMAUCHI, Kyoto (JP); Tsutomu ICHIKAWA, Kyoto (JP)

(21) Appl. No.: 17/152,254

(22) Filed: Jan. 19, 2021

Related U.S. Application Data
(63) Continuation of application No. PCT/JP2019/026080, filed on Jul. 1, 2019.

(30) Foreign Application Priority Data
Jul. 26, 2018 (JP) 2018-140347

(51) Int. Cl. A61B 5/024; A61B 5/00

(52) U.S. Cl. CPC A61B 5/02438; A61B 5/0022; A61B 5/7405; A61B 5/7455; A61B 5/742

(57) ABSTRACT

An information processing device in a biometric data measurement system according to a first embodiment of the present invention includes a communication unit, a display unit including a display screen, a connection unit connecting to the biometric data measurement device, an operation unit receiving a start operation for a measurement operation for biometric data, and a control unit transmitting a measurement start command for starting measurement of the biometric data, to the biometric data measurement device via the connection unit when a measurement start condition is satisfied after the operation section receives the start operation, and the measurement start condition includes prevention of the subject from viewing a display content of the display screen.

### TECHNICAL FIELD

The present invention relates to a biometric data measurement system and an information processing device.

### BACKGROUND ART

In recent years, devices measuring various biometric data such as blood pressure, electrocardiographic data, and the like have been used at home. One such biometric data measurement device is an electronic blood pressure monitor. A common electronic blood pressure monitor includes a cuff wrapped around the upper arm or the wrist, and a body connected with the cuff integrally or via a tube. The following are disposed in the body: a mechanical structure including a pump for feeding a fluid (typically air) to a fluid bag provided in the cuff, a pressure sensor for detecting a pressure change in the fluid, and the like, and an electrical circuit controlling a pump and the like, calculating a blood pressure value based on a pressure sensor output. A body surface is provided with user interfaces including an operation member for instructing operation of an electronic blood pressure monitor and a display unit for displaying the calculated blood pressure value.

On the other hand, as in Patent Documents 1 and 2, a biometric data measurement system has been proposed in which the main user interfaces such as the operation member and the display unit are omitted from the body, or which includes a minimum necessary user interface mounted on the body and utilizes a user interface provided as an application program for an information processing device such as a smartphone.

### CITATION LIST

### Patent Literature

Patent Document 1: JP 6251239 B
Patent Document 2: US 2014/0,159,912 A

### SUMMARY OF INVENTION

### Technical Problem

The information processing device can execute various application programs, and some of the application programs, for example, messaging application programs, are constantly executed in the background. For example, when a message is received, a messaging application program emits a ringtone or a ringer melody through a speaker, changes a pictogram on an upper part of a display screen, or displays a message content in a pop-up window on the display screen.

Thus, even in a biometric data measurement system in which the user interface is an information processing device, as disclosed in Patent Documents 1 and 2, some application programs are executed in the background even during measurement of biometric data such as the blood pressure. Thus, depending on the content displayed on the display screen of the information processing device by the application program during measurement of biometric data, the mental state may change to deteriorate the resting state, affecting the biometric data such that, for example, the blood pressure increases. This may prevent accurate biometric data measurement.

In light of the above-described circumstances, and an object of the present invention is to provide a biometric data measurement system and the information processing device that can perform accurate biometric data measurement utilizing an information processing device as a user interface.

### Solution to Problem

The present invention adopts the following configurations in order to achieve the above-mentioned object.

In one aspect of the invention, a biometric data measurement system includes:

a biometric data measurement device configured to measure biometric data of a subject; and
an information processing device configured to control a biometric data measurement operation by the biometric data measurement device, wherein
the information processing device includes:
a communication unit configured to connect to a communication network;
a display unit including a display screen displaying various pieces of information;
a connection unit configured to connect to the biometric data measurement device wirelessly or by wire;
an operation unit configured to receive a start operation for a measurement operation for biometric data of the subject by the biometric data measurement device; and
a control unit configured to transmit a measurement start command for starting measurement of the biometric data, to the biometric data measurement device via the connection unit when a measurement start condition is satisfied after the operation section receives the start operation, and
the measurement start condition includes prevention of the subject from viewing a display content of the display screen. Note that when the measurement start condition is satisfied, the control unit may further erase a display screen of the display unit. According to this configuration, the biometric data measurement is started under a condition that the subject is prevented from viewing the display content of the display screen. Thus, regardless of whatever content is displayed by the application program executed by the information processing device in the background during the biometric data measurement, the subject is prevented from viewing the content, preventing the biometric data measurement from being affected. Consequently, even in a case where the information processing device is used as a user interface, accurate biometric data measurement can be performed. When the measurement start condition is satisfied, erasing the display screen of the display unit enables suppression of power consumption of the information processing device.

In one aspect of the invention, the information processing device may further include a sensor configured to detect an angle of the display screen with respect to a gravitational direction, and the control unit may include a determination unit configured to determine that the measurement start condition is satisfied when the angle of the display screen equals a measurement start angle at which the subject is prevented from viewing the display screen. For example, a sensor such as an acceleration sensor or a gyroscopic sensor can be used to detect the angle of the display screen, that is, the direction in which the display screen faces. The determination unit may determine that the measurement start condition is satisfied, for example, when the display screen is oriented approximately in the ground direction, that is, when the information processing device is laid face down on a desk or the like. The start operation of the subject acting as an operator operating the information processing device can be performed, for example, by touching an icon or button image on the display screen, and of course, the subject needs to be able to view the display screen. In a case where the subject performs the start operation with the information processing device in hand rather than with the information processing device placed on a desk or the like, the display screen generally substantially directly faces the face of the subject. Thus, the measurement start angle can be an angle greater than ±90 degrees with respect to the angle of the display screen of the display unit when the subject receives the start operation.

According to this configuration, the use of the angle of the display screen as an indicator allows the subject as the operator to be easily determined to be prevented from viewing the display content of the display screen.

In the aspect described above, the information processing device may further include a sensor configured to detect that the display screen is covered, and the control unit may include a determination unit configured to determine that the measurement start condition is satisfied when the display screen is covered. For example, in a case where the information processing apparatus is in a notebook type cover, closing the cover covers the display screen to allow no one to view the display screen. When the information processing device is laid face down on a desk or the like, the display screen is covered by a top plate of the desk. For example, the illuminance of the display screen can be detected by a sensor such as a front camera (sub-camera) or an illuminance sensor provided on the same surface as the surface on which the display screen of the information processing device is disposed. The determination unit can determine whether the display screen is covered based on the illuminance detected by the sensor. According to this configuration, by using, as an indicator, whether the display screen is covered, the subject can be easily determined to be prevented from viewing the display content of the display screen.

In the aspect described above, the information processing device may further include a sensor configured to detect a line of sight of an operator operating the operation unit, and the control unit may include a determination unit configured to determine that the measurement start condition is satisfied when the line of sight of the operator is away from the display screen. When the subject acting as an operator operating the information processing device performs the start operation, the subject naturally views the display screen. Thus, for example, in a case where the line of sight of the subject is detected by a known line-of-sight detection technique utilizing a sensor such as a front camera (sub-camera) provided on the same surface as the surface on which the display screen of the information processing device is placed and where the detected line of sight is away from the display screen, the subject can be determined to be prevented from viewing the display content of the display screen. Thus, the determination unit can determine whether the line of sight of the subject is away from the display screen based on an image captured by a sensor such as a front camera. According to this configuration, by using, as an indicator, whether the line of sight of the subject is away from the display screen, the subject can be easily determined to be prevented from viewing the display content of the display screen.

In the aspect described above, the information processing device may further include a sensor configured to detect that the biometric data measurement device is present opposite to the display screen with respect to the information processing device, and the information processing device may include a determination unit configured to determine, when the operation unit receives the start that the measurement start condition is satisfied in a case where the biometric data measurement device is present opposite to the display screen with respect to the information processing device. In a case where the operator operating the information processing device is different from the subject of the biometric data, the positional relationship between the subject and the information processing device varies, and it is difficult to determine which orientation of the display screen prevents the subject from viewing the display content of the display screen. However, in a case where the biometric data measurement device is present opposite to the display screen when the start operation is performed, the subject wearing the biometric data measurement device is reliably opposite to the display screen. Thus, this can be used as an indicator. For example, in a case where a sensor such as a rear camera (main camera) typically provided in the information processing device is utilized to extract, from an image, a logo of the product name of the biometric data measurement device, a model number, or the like and where the logo or the model number corresponds to a particular or preset biometric data measurement device, the determination unit can determine that the biometric data measurement device is present opposite to the display screen. According to this configuration, whether a biometric data measurement device is present opposite to the display screen or not is used as an indicator. This allows the subject to be easily determined to be prevented from viewing the display content of the display screen.

In the aspect described above, the control unit may control the display unit such that when the operation unit receives the start the display unit changes display on the display screen to a display state in which the subject is prevented from viewing the display, and the control unit may include a determination unit configured to determine that the measurement start condition is satisfied when the display content of the display screen changes to the display state in which the subject is prevented from viewing the display content. For example, the control unit forcibly dims the display screen, preventing the subject from viewing the display content of the display screen. However, sudden erasure of the display screen may be mistaken for a failure or may surprise the subject, adversely affecting the biometric data measurement. Thus, the display on the display screen is desirably gradually dimmed. Alternatively, the control unit may forcibly activate a screen saver.

According to this configuration, by using, as an indicator, whether the display on the display screen has changed to a state in which the subject is prevented from viewing the display content, the subject can be easily determined to be prevented from viewing the display content of the display screen.

In the aspect described above, desirably, the information processing device further includes a notification unit including at least one of a speaker configured to generate sound and a vibration member configured to generate vibration, and the control unit causes, after elapse of a certain period of time since satisfaction of the measurement start condition, the notification unit to notify the subject of start of measurement, and the control unit transmits, after notification of the start of measurement by the notification unit, the measurement start command to the biometric data measurement device via the connection unit.

According to this configuration, when the measurement start condition is satisfied, after the elapse of a certain period of time since the satisfaction, the start of measurement is notified and then the measurement is started. Thus, as is the case with a typical biometric data measurement device, the subject can behave from sensing of the start of measurement. Because the notification is provided in a case where the subject is prevented from viewing the display content of the display screen, the notification of the start of measurement is provided by sound or vibration rather than being displayed on the display screen.

In the aspect described above, the control unit may prohibit operation of the notification unit during measurement of the biometric data by the biometric data measurement device, and when a measurement result for the biometric data is received from the biometric data measurement device via the connection unit, the control unit may cause the notification unit to notify the subject of end of the measurement. According to this configuration, during the biometric data measurement, sound or vibration that inhibits the resting state of the subject, such as a reception ringtone or alert vibration is prevented from being produced during message reception. This enables a further reduction in the adverse effect of the operation of another application program. When the measurement result is received from the biometric data measurement device, the end of measurement is notified by sound or vibration, and thus the end of the biometric data measurement can be reliably notified to the subject.

In the aspect described above, the control unit may include an operation guidance unit configured to display a guidance message prompting movement of the information processing device to satisfy the measurement start condition, on the display screen when the operation unit receives the start operation.

According to this configuration, by displaying and/or audibly outputting, during the start operation, the guidance message prompting movement of the information processing device to satisfy the measurement start condition, how to actually start the measurement can be indicated to the subject. Furthermore, the guidance message may be audibly output.

In the aspect described above, the control unit may include a notification unit configured to display a message promoting biometric data measurement with the biometric data measurement device, on the display screen at a preset date and time, and the control unit may gradually dim the display on the display screen after the notification unit outputs the message. Note that, when the control unit gradually dims display in such manner, in a case where the control unit is connected to the biometric data measurement device within a certain period of time, the control unit preferably recovers the display on the display screen to an original state and then starts to determine whether the measurement start condition is satisfied. Furthermore, the message prompting the biometric data measurement with the biometric data measurement device may be audibly output.

According to this configuration, at or near the date and time of start of the measurement, the message prompting the biometric data measurement with the biometric data measurement device is displayed and/or audibly output. Thus, the subject can be prompted to measure the biometric data at a preset date and time and prevented from forgetting the measurement. Moreover, when the display is gradually dimmed after the message prompting the biometric data measurement is output, even in a case where the subject is operating any other application program on the information processing device, the operation is made difficult, allowing the subject to be diverted to the biometric data measurement.

In the aspect described above, the biometric data measurement device can include a blood pressure monitor that includes: a blood pressure measurement unit including a cuff containing a fluid bag and wrapped around a measured site of the subject and configured to measure a blood pressure of the subject; a detection unit configured to detect a state of wrapping, around the measured site, of the cuff of the blood pressure measurement unit; a blood pressure monitor connection unit connected wirelessly or by wire to the information processing device, and a blood pressure monitor control unit configured to cause the blood pressure measurement unit to measure the blood pressure of the subject when the measurement start command is received from the information processing device via the blood pressure monitor connection unit, and transmit a result of the measurement to the information processing device via the blood pressure monitor connection unit.

According to this configuration, the blood pressure monitor, corresponding to the biometric data measurement device, can start measurement of the blood pressure in response to the operation of the information processing device by the subject, allowing the operation unit to be omitted from the blood pressure monitor. Furthermore, the blood pressure measurement result from the blood pressure monitor can be transmitted to the information processing device and displayed on the information processing device side, allowing the display unit to be omitted from the blood pressure monitor.

Here, the control unit may include an inquiry unit configured to inquire of the blood pressure monitor, via the connection unit, whether or not preparation for measurement with the blood pressure measurement unit is completed, and when the inquiry is received from the information processing device, the blood pressure monitor control unit may transmit a measurement preparation completion notification to the information processing device via the blood pressure monitor connection unit in a case where a state of wrapping of the cuff around the measured site detected by the detection unit corresponds to a blood pressure measurement enabled state, and when the measurement preparation completion notification is received, the control unit may start determining whether the measurement start condition is satisfied.

According to this configuration, in response to the start operation, whether the blood pressure monitor is ready is checked before determination of whether the measurement start condition is satisfied is started. This allows prevention of possible start of blood pressure measurement before a state required for measurement of the blood pressure of the subject is established. Thus, even in a case where the operation member is separate from the blood pressure monitor, the measurement can be made reliable, while the safety of the measurement can be ensured.

When the measurement start condition is no longer satisfied after the measurement start command is transmitted to the blood pressure monitor, the control unit may transmit a measurement stop command to the blood pressure monitor via the connection unit, and when the blood pressure monitor control unit receives the measurement stop command from the information processing device, in a case where a compressed state by the fluid bag contained in the cuff of the blood pressure measurement unit continues for a certain period of time or longer, the blood pressure monitor control unit may discontinue the measurement of the blood pressure and causes a fluid in the fluid bag to be discharged. For example, in the sense of a switch for emergency discharge (stop) in preparation for a case where an abnormality occurs in a fluid feeding mechanism of the blood pressure monitor and the compressed state by the fluid bag continues, “in a case where the compressed state continues for longer than a certain period of time and the information processing device in, for example, a face-down state is suddenly moved,” processing for emergency stop can be executed. According to this configuration, when the measurement start condition is no longer satisfied, the blood pressure measurement is discontinued and the fluid in the fluid bag is discharged in a case where the compressed state by the fluid bag of the cuff continues for a certain period of time or longer. Thus, even in a case where the operation member is separate from the blood pressure monitor, the safety can be ensured.

The blood pressure monitor may further include an emergency stop button dedicated to emergency stop or also used as another button, and in response to an operation of the emergency stop button, the blood pressure monitor control unit may discontinue the measurement of the blood pressure and cause the fluid in the fluid bag contained in the cuff of the blood pressure measurement unit to be discharged. According to this configuration, the blood pressure monitor discontinues the blood pressure measurement and causes the fluid in the fluid bag to be discharged in response to an operation of the emergency stop button provided in the blood pressure monitor, and thus the safety of the measurement can be ensured.

In another aspect of the present invention, an information processing device includes

a communication unit configured to connect to a communication network;
a display unit including a display screen displaying various pieces of information;
a connection unit configured to connect to another device wirelessly or by wire;
an operation unit configured to receive a user operation; and
a control unit configured to control each of the units, wherein the control unit
determines whether a measurement start condition is satisfied when the operation unit receives a measurement start operation for a biometric data measuring device measuring biometric data of a subject, and
transmits a measurement start command to the biometric data measurement device via the connection unit in a case where the measurement start condition is satisfied, and the measurement start condition includes prevention of the subject from viewing a display content of the display screen. Note that when the measurement start condition is satisfied, the control unit may further erase a display screen of the display unit.

According to this configuration, the information processing device instructs the biometric data measurement device to start biometric data measurement provided that the subject is prevented from viewing the display content of the display screen. Thus, regardless of whatever content is displayed by the application program executed by the information processing device in the background during the biometric data measurement, the subject is prevented from viewing the content, preventing the biometric data measurement by the biometric data measurement device from being affected. Consequently, even in a case where the information processing device is used as a user interface, accurate biometric data measurement can be performed. When the measurement start condition is satisfied, the display screen of the display unit is further erased, enabling power consumption of the information processing device to be suppressed.

In another aspect of the present invention, a method for causing a biometric data measurement device to measure biometric data is a method for causing a biometric data measurement device to measure biometric data, the method being executed by an information processing device including a processor, a memory, a communication unit connecting to a communication network, a display unit including a display screen displaying various pieces of information, a connection unit connecting to another device wirelessly or by wire, and an operation unit receiving a user operation, the method including the steps of:

the processor determining whether a measurement start condition is satisfied when the operation unit receives a measurement start operation for the biometric data measuring device, and
the processor transmitting a measurement start command to the biometric data measurement device via the connection unit in a case where the measurement start condition is satisfied,
wherein the measurement start condition includes prevention of the subject of the biometric data from viewing a display content of the display screen.

According to this configuration, the information processing device instructs the biometric data measurement device to start biometric data measurement on condition that the subject is prevented from viewing the display content of the display screen. Thus, regardless of whatever content is displayed by the application program executed by the information processing device in the background during the biometric data measurement, the subject is prevented from viewing the content, preventing the biometric data measurement by the biometric data measurement device from being affected. Consequently, even in a case where the information processing device is used as a user interface, accurate biometric data measurement can be performed.

In another aspect of the invention, a biometric data measurement program may be a program causing a processor to function as the control unit included in the information processing device according to the aspect described above or another aspect.

According to this configuration, the information processing device including the processor executing the biometric data measurement program instructs the biometric data measurement device to start biometric data measurement on condition that the subject is prevented from viewing the display content of the display screen. Thus, regardless of whatever content is displayed by the application program executed by the information processing device in the background during the biometric data measurement, the subject is prevented from viewing the content, preventing the biometric data measurement by the biometric data measurement device from being affected. Consequently, even in a case where the information processing device is used as a user interface, accurate biometric data measurement can be performed.

### Advantageous Effects of Invention

According to the present invention, a technique can be provided that allows accurate biometric data measurement to be performed even in a case where an information processing device is used as a user interface.

### BRIEF DESCRIPTION OF DRAWINGS

FIG. 1 is a diagram schematically illustrating an example of a biometric data management system according to an embodiment.

FIG. 2 is a block diagram schematically illustrating an example of a configuration of a blood pressure monitor and an information processing device in a biometric data measurement system according to the embodiment.

FIG. 3 is a block diagram illustrating an example of a configuration of a blood pressure monitor in a biometric data measurement system according to a first embodiment.

FIG. 4 is a block diagram illustrating an example of a configuration of an information processing device in the biometric data measurement system according to the first embodiment.

FIG. 5 is a flowchart illustrating an example of a processing procedure of the information processing device in accordance with a blood pressure measurement application program.

FIG. 6A illustrates a first portion of the flowchart illustrating an example of a processing procedure for a blood pressure monitor.

FIG. 6B illustrates a second portion of the flowchart illustrating an example of the processing procedure for the blood pressure monitor.

FIG. 7 is a flowchart illustrating an example of a processing procedure of measurement start determination processing in FIG. 5.

FIG. 8 is a flowchart illustrating an example of a processing procedure of measurement start determination processing in a first modified example of the first embodiment.

FIG. 9 is a flowchart illustrating an example of a processing procedure of measurement start determination processing in a second modified example of the first embodiment.

FIG. 10 is a flowchart illustrating an example of a processing procedure of measurement start determination processing in a third modified example of the first embodiment.

FIG. 11A is a flowchart illustrating an example of a processing procedure of measurement start determination processing in a fourth modified example of the first embodiment.

FIG. 11B is a flowchart illustrating another example of the processing procedure of measurement start determination processing in the fourth modified example of the first embodiment.

FIG. 12 is a flowchart illustrating an example of a processing procedure for an information processing device in accordance with a blood pressure measurement application program in a biometric data measurement system according to a second embodiment.

### DESCRIPTION OF EMBODIMENTS

Embodiments of the present invention will be described below based on the drawings.

Here, as an example, a case will be described in which a biometric data measurement device is an electronic blood pressure monitor that uses a technique (oscillometric method) for compressing the blood vessel to detect a pulse wave.

### Application Example

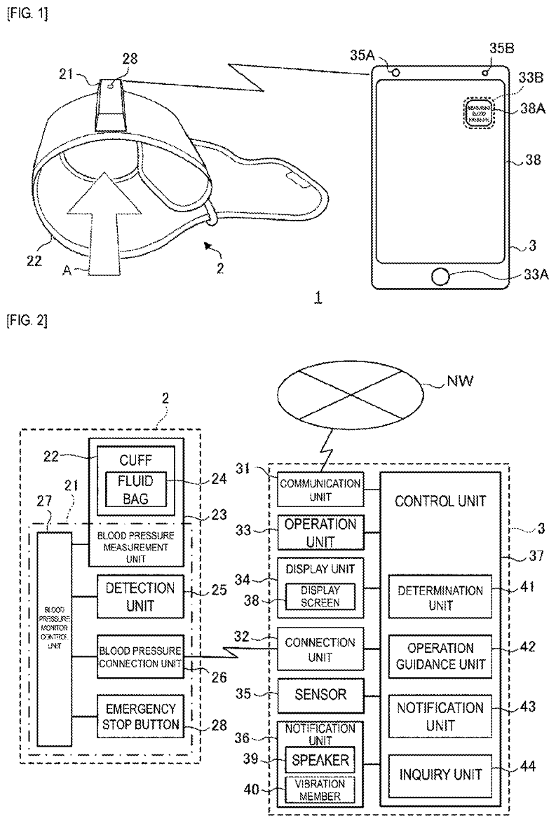

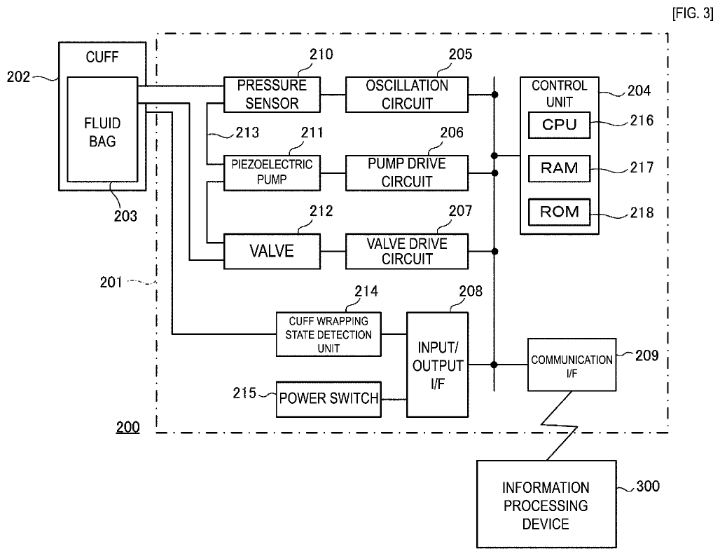

First, with reference to FIGS. 1 and 2, an example of a case in which the present invention is applied will be described. FIG. 1 schematically illustrates an example of a biometric data measurement system 1 according to an embodiment. FIG. 2 schematically illustrates an example of the configuration of the biometric data measurement system 1.

### Configuration of Application Example

In the example in FIGS. 1 and 2, the biometric data measurement system 1 includes a blood pressure monitor 2 that measures the blood pressure of a subject, and an information processing device 3 that controls a blood pressure measurement operation by the blood pressure monitor 2.

The blood pressure monitor 2 is, for example, an electronic blood pressure monitor including a blood pressure monitor body 21 and a cuff 22 wrapped around a measured site of a subject, such as the upper arm or wrist of the subject.

The cuff 22 is a part of the blood pressure measurement unit 23 that measures the blood pressure of the subject and contains, for example, a fluid bag 24. Other portions of the blood pressure measurement unit 23 are configured in the blood pressure monitor body 21. The blood pressure measurement unit 23 may include, in the blood pressure monitor body 21, for example, a piezoelectric pump, a pressure sensor, a valve, or the like.

In addition to a part of the blood pressure measurement unit 23, the blood pressure monitor body 21 includes, for example, a detection unit 25, a blood pressure monitor connection unit 26, and a blood pressure monitor control unit 27. Although not illustrated, the blood pressure monitor body 21 includes, for example, a battery and a power button. The blood pressure monitor body 21 may further include an emergency stop button 28, for example.

The detection unit 25 detects a state of wrapping of the cuff 22 around the upper arm, wrist, or the like, corresponding to the measured site of the subject. The detection unit 25 may detect the state of wrapping of the cuff 22 by detecting, for example, a state of electrical connection of an electrical contact provided in the cuff 22, marks aligned along the longitudinal direction of the cuff 22 and optically detected, a wrapping shape of the cuff 22 detected by a shape sensor such as a fiber sensor inserted through the cuff 22 in the longitudinal direction, and the like.

The blood pressure monitor connection unit 26 is a communication interface that connects to the information processing device 3 wirelessly or by wire and transmits and receives information to and from the information processing device 3. The blood pressure monitor connection unit 26 may be, for example, a near field wireless communication interface. The near field wireless communication interface may be, but is not limited to, a BLE communication module employing near field wireless data communication standards such as, for example, Bluetooth (trade name). The blood pressure monitor connection unit 26 may be a wired communication interface such as a Universal Serial Bus (USB) interface that communicates via a Universal Serial Bus (USB) cable.

Although not illustrated, the blood pressure monitor control unit 27 includes, for example, a processor such as a central processing unit (CPU) or a micro processing unit (MPU) and a memory such as a random access memory (RAM), a read only memory (ROM), and the like, and controls components of the blood pressure monitor 2 according to information processing. The ROM stores, for example, programs to be executed by the processor. The RAM is, for example, a memory used as a work memory by the processor. By executing the programs required by the processor, the blood pressure monitor control unit 27 can perform various operations.

The emergency stop button 28 is a button for instructing the blood pressure monitor control unit 27 to stop the operation of the blood pressure monitor 2, for example. Note that the emergency stop button 28 is not limited to a dedicated button, and may also be used as another button such as a power button. For example, in a case where the emergency stop button is also used as another button, the usage is assumed in which, for example, power on/off is instructed by long depression, whereas emergency stop is instructed by short depression.

The information processing device 3 may be a smart device such as a typical smartphone or a tablet terminal. The information processing device 3 includes, for example, a communication unit 31, a connection unit 32, an operation unit 33 including an operation button 33A, a display unit 34, a sensor 35, a notification unit 36, and a control unit 37.

The communication unit 31 may include, for example, a Wi-Fi (trade name) module. The communication unit 31 is, for example, connected to a communication network NW such as the Internet or a telephone network via a Wi-Fi base station not illustrated, and communicates with an external device via the communication network NW.

The connection unit 32 is a communication interface that connects wirelessly or by wire to the blood pressure monitor connection unit 26 of the blood pressure monitor 2 and that transmits and receives information to and from the blood pressure monitor 2. The connection unit 32 may be, for example, a near field wireless communication interface. The near field wireless communication interface may be, but is not limited to, a BLE communication module employing near field wireless data communication standards such as, for example, Bluetooth (trade name). The connection unit 32 may be a wired communication interface, for example, a USB interface that communicates via a USB cable.

The operation unit 33 includes various operation buttons, for example, an operation button 33A provided on a front surface of the information processing device 3 and an operation button (not illustrated) provided on a side surface of the information processing device 3. The operation unit 33 includes a touch panel disposed on a display screen of the display unit 34, for example.

The display unit 34 includes a display screen 38 that displays various pieces of information including information related to measurement, for example. The information related to measurement may include, for example, a measurement start guidance message, an error message, a measurement result, and the like. The display screen 38 may be, for example, a liquid crystal display or an organic electroluminescent display.

The sensor 35 may include, for example, a sensor that detects the angle of the display screen 38 with respect to the gravitational direction, a sensor that detects that the display screen 38 is covered, a sensor that detects that the line of sight of an operator operating the operation unit is away from the display screen 38, a sensor detecting that the blood pressure monitor 2 is present opposite to the display screen 38, and the like.

The sensor that detects the angle of the display screen 38 with respect to the gravitational direction includes, for example, an acceleration sensor and a gyroscopic sensor. The acceleration sensor detects, for example, the acceleration of the information processing device 3. The acceleration sensor is not limited to detection of the accelerations in the directions of the X, Y, and Z axes, for example, and may detect the acceleration in the rotational direction around each axis. The gyroscopic sensor detects the angle of rotation in a case where the information processing device 3 rotates around each of the X, Y, and Z axes.

The sensor that detects that the display screen 38 is covered includes, for example, a front camera 35A and an illuminance sensor 35B provided on the same face as a surface on which the display screen 38 of the information processing device 3 is disposed (hereinafter, referred to as the front surface). The front camera 35A is, for example, a camera disposed in proximity to the display screen 38 on the front surface of the information processing device 3, and can capture an image of, for example, the face of the operator operating the information processing device 3. The illuminance sensor 35B is disposed, for example, on the front surface of the information processing device 3 in proximity to the display screen 38, and detects the illuminance on the display screen 38.

The sensor detecting that the line of sight of the operator operating the operation unit 33 is away from the display screen 38 can include, for example, the front camera 35A.

The sensor detecting that the blood pressure monitor 2 is present opposite to the display screen 38 includes, for example, a rear camera disposed on a surface of the information processing device 3 opposite to the front surface of the information processing device 3 (hereinafter referred to as the rear face). Typically, the rear camera used provides a higher definition and higher functions than the front camera 35A. Thus, the rear camera is also referred to as a main camera, and the front camera 35A may be referred to as a sub-camera.

The notification unit 36 may include a speaker 39 and a vibration member 40. The speaker 39 produces sound and, together with a microphone not illustrated, functions as a user interface for a phone call. In addition to telephone voice, the speaker 39 can output various sounds, such as ringtones for telephone, email, messages, and music. The vibration member 40 is, for example, a vibrator that vibrates the information processing device 3, for example, when a telephone call, an email, a message, or the like is received. The vibration member 40 may be used, for example, in order to provide a realistic sensation during the execution of a game application program in the information processing device 3.

Although not illustrated in the drawings, the control unit 37 includes a processor such as a CPU or an MPU and a memory such as a RAM and a ROM, for example, and controls the components of the information processing device 3 in accordance with information processing. The ROM stores, for example, programs to be executed by the processor. The RAM is, for example, a memory used as a work memory by the processor. With the processor executing the required program, the control unit 37 can perform various operations.

The control unit 37 can perform functions of, for example, a determination unit 41, an operation guidance unit 42, a notification unit 43, and an inquiry unit 44. The determination unit 41 determines, for example, whether a start condition for blood pressure measurement performed by the blood pressure monitor 2 is satisfied or not. For example, when the operation unit 33 receives the start operation, the operation guidance unit 42 displays, on the display screen 38, a guidance message prompting movement of information processing device 3 to satisfy the measurement start condition. The notification unit 43, for example, displays a message prompting blood pressure measurement with the blood pressure monitor 2, on the display screen 38 at a preset date and time. The inquiry unit 44, for example, inquiries of the blood pressure monitor 2 via the connection unit 32, whether measurement preparation for the blood pressure measurement unit 23 is complete.

### Operation of Application Example

Now, an example of the operation of the biometric data measurement system 1 will be described.

In a case where the subject measures the blood pressure, the wrist is passed through the cuff 22, as indicated by arrow A in FIG. 1, with the palm of the subject facing upward, to attach the blood pressure monitor 2 to the wrist. Before or after this attachment operation, an operator who is a subject operates a power switch (not illustrated) of the blood pressure monitor 2, for example, to make the blood pressure monitor 2 operable. The subject activates the blood pressure measurement application program of the pre-paired information processing device 3, for example. Thus, an operation is performed that establishes communication between the blood pressure monitor connection unit 26 of the blood pressure monitor 2 and the connection unit 32 of the information processing device 3. When the blood pressure measurement application program of the information processing device 3 is activated, the control unit 37 of the information processing device 3, for example, causes the display screen 38 of the display unit 34 to display an icon 38A for instructing the start of blood pressure measurement.

In such a situation, when the operator corresponding to the subject touches the icon 38A, a touch sensor 33B at the touch position is turned on on a touch panel on the display screen 38 included in the operation unit 33 of the information processing device 3. The operation unit 33 outputs coordinate data indicating the touch position to the control unit 37, for example. The control unit 37 can determine, for example, from the display content of the display screen 38 at the touch position, that a start operation has been performed that starts a blood pressure measurement operation by the blood pressure monitor 2. In this way, the operation unit 33 receives the start operation for the blood pressure measurement operation by the blood pressure monitor 2.

After receiving the start operation from the operation unit 33, the control unit 37, for example, causes the determination unit 41 to determine whether a measurement start condition is satisfied. The measurement start condition includes, for example, prevention of the subject from viewing the display content of the display screen 38 of the display unit 34. Then, when the measurement start condition is satisfied, the control unit 37, for example, causes the connection unit 32 to transmit a measurement start command to the blood pressure monitor 2. The control unit 37 may, for example, further erases the display screen 38 of the display unit 34 when the measurement start condition is satisfied. Erasing the display screen 38 allows power consumption of the information processing device 3 to be suppressed.

For example, after communication is established between the blood pressure monitor connection unit 26 and the connection unit 32 of the information processing device 3, or after connection is complete, the blood pressure monitor 2 waits to receive the measurement start command from the information processing device 3. When the measurement start command is received from the information processing device 3 through the blood pressure monitor connection unit 26, the blood pressure monitor control unit 27, for example, causes the blood pressure measurement unit 23 to measure the blood pressure of the subject, and transmits a measurement result to the information processing device 3 through the blood pressure monitor connection unit 26.

For example, after causing the connection unit 32 to transmit the measurement start command to the blood pressure monitor 2, the information processing device 3 waits to receive the measurement result from the blood pressure monitor 2. When the information processing device 3 receives a measurement result from the blood pressure monitor 2 through the connection unit 32, the control unit 37, for example, displays the received measurement result on the display screen 38 of the display unit 34. The control unit 37 can cause, for example, the communication unit 31 to transmit the received measurement result to an external device, for example, an information terminal of a physician or a server device via the communication network NW.

In this way, the control unit 37 of the information processing device 3 can cause the blood pressure measurement with the blood pressure monitor 2 to be started, for example, on condition that the subject is prevented from viewing the display content of the display screen 38. Thus, regardless of whatever content is caused to be displayed on the display screen by an application program executed by the information processing device 3 in the background during the blood pressure measurement, the subject is prevented from viewing the content, thus preventing the blood pressure measurement from being affected. Accordingly, even in a case where the information processing device 3 is used as a user interface for the blood pressure monitor 2, accurate blood pressure measurement can be performed.

Note that during measurement by the blood pressure monitor 2, for example, the control unit 37 prohibits the operation of the speaker 39 and the vibration member 40 included in the notification unit 36, and performs control to prevent possible sound or vibration that inhibits the resting state of the subject, such as a reception ringtone or alert vibration during message reception. Thus, even with an incoming email or message, not only is the display invisible but also no ringtone or vibration is produced, enabling a further reduction in the adverse effect of operation of another application program such as an email application program or a messaging application program. Furthermore, the control unit 37 may, for example, perform control to put on, through the speaker 39, music or the like that assists in maintaining the resting state of the subject during the blood pressure measurement.

Instead of immediately prohibiting the operation of the speaker 39 and the vibration member 40 of the notification unit 36 when the measurement start condition is satisfied, the notification unit 36 may produce a ringtone or alert vibration that notifies the start of measurement a certain period of time after the measurement start condition is satisfied. Then, after the notification, the connection unit 32 transmits the measurement start command to the blood pressure monitor 2 to prohibit the operation of the notification unit 36. In this manner, when the measurement start condition is satisfied, the start of measurement is notified a certain period of time after the satisfaction, before the measurement is started. This enables the subject to behave from sensing of the start of measurement, as is the case with typical blood pressure monitors.

Then, when the measurement result is received from the blood pressure monitor 2 through the connection unit 32, the control unit 37 may notify the end of measurement using a ringtone produced by the speaker 39 of the notification unit 36 or vibration caused by the vibration member 40 of the notification unit 36. In this way, when the measurement result is received from the blood pressure monitor 2, the end of measurement is notified by the ringtone or vibration, thus allowing the end of the blood pressure measurement to be reliably notified to the subject.

When the operation unit 33 receives the start operation, the control unit 37 may perform, for example, an operation described below instead of immediately starting the determination of whether the measurement start condition is satisfied. In other words, for example, when the operation unit 33 receives the start operation, the inquiry unit 44 of the control unit 37 can inquire of the blood pressure monitor 2 via the connection unit 32 whether measurement preparation for the blood pressure measurement unit 23 is complete. For example, when an inquiry is received from the information processing device 3 through the blood pressure monitor connection unit 26, the blood pressure monitor control unit 27 detects the state of wrapping of the cuff 22 of the blood pressure measurement unit 23 around the measured site, and in a case where the state of wrapping is determined to enable the blood pressure measurement, transmits a measurement preparation completion notification to the information processing device 3 through the blood pressure monitor connection unit 26. The control unit 37 starts determining whether the measurement start condition is satisfied, for example, when the connection unit 32 receives the measurement preparation completion notification. This eliminates the possibility that the blood pressure measurement is started before the state required to measure the blood pressure of the subject is established, and even in a case where the operation member is separate from the blood pressure monitor 2, the measurement can be made reliable, while the safety of the measurement can be ensured.

For example, when the operation unit 33 receives the start operation, the operation guidance unit 42 of the control unit 37 may display, on the display screen 38 of the display unit 34, a guidance message prompting movement of the information processing device 3 to satisfy the measurement start condition, or audibly output the guidance message through the speaker 39. In this way, how to actually start the measurement can be indicated to the subject.

Note that the measurement start condition, used as a reference for the determination unit 41 of the control unit 37, may be that, for example, the angle of the display screen 38 of the display unit 34 equals a measurement start angle at which the subject is prevented from viewing the display screen 38. For example, the sensor 35 such as an acceleration sensor or a gyroscopic sensor can detect the angle of the display screen 38 with respect to the gravitational direction, that is, the direction in which the display screen 38 faces. Thus, for example, when the display screen 38 is at an angle at which the display screen 38 approximately faces the ground direction, that is, the gravitational direction, the information processing device 3 can be determined to be laid face down on a desk or the like, and this state may be determined to indicate that the angle of the display screen 38 equals the measurement start angle. The start operation of the operator can also be performed, for example, by touching an icon or a button image on the display screen 38, and of course the operator needs to be able to view the display screen 38. In a case where the operator performs the start operation with the information processing device 3 in hand rather than with the information processing device 3 placed on a desk or the like, the display screen 38 generally substantially directly faces the face of the subject. Consequently, the measurement start angle may be, for example, an angle exceeding a predetermined angle with respect to the angle of the display screen 38 when the operator performs the start operation, for example, an angle exceeding the range of ±90 degrees from the angle of the display screen 38. Thus using the angle of the display screen 38 as an indicator allows the subject to be easily determined to be prevented from viewing the display content of the display screen 38.

Alternatively, the measurement start condition, used as a reference of the determination unit 41 of the control unit 37, may be, for example, that the display screen 38 of the display unit 34 is covered. For example, in a case where the information processing device 3 is placed in a notebook type cover, closing the cover covers the display screen 38 to prevent the subject from viewing the display screen 38. When the information processing device 3 is laid face down on a desk or the like, the display screen 38 is covered by a top plate of the desk. Whether the display screen 38 of the display unit 34 is covered may be determined, for example, based on the illuminance of the display screen 38 detected by a sensor 35 such as the illuminance sensor 35B or the front camera 35A provided on the front surface of the information processing device 3. In this way, by using, as an indicator, whether the display screen 38 is covered, the subject can be easily determined to be prevented from viewing the display content of the display screen 38.

The measurement start condition, used as a reference for the determination unit 41 of the control unit 37, may be, for example, that the line of sight of the operator operating the operation unit 33 is away from the display screen 38 of the display unit 34. When the operator performs the start operation, the operator views the display screen 38. Thus, for example, the line of sight of the operator is detected by a known line-of-sight detection technique utilizing the sensor 35 such as the front camera 35A of the information processing device 3, and in a case where the line of sight detected is away from the display screen, it is determined that the operator is prevented from viewing the display content of the display screen 38. Consequently, for example, whether the line of sight of the operator is away from the display screen 38 of the display unit 34 can be determined based on an image captured by the front camera 35A. In this way, by using, as an indicator, whether the line of sight of the subject corresponding to the operator is away from the display screen 38, the subject can be easily determined to be prevented from viewing the display content of the display screen 38.

The measurement start condition, which is a reference of the determination unit 41 of the control unit 37, may be that, for example, when the operation unit 33 receives the start operation, the blood pressure monitor 2 is present at a rear surface of the information processing device 3 opposite to the display screen 38 of the display unit 34. In a case where the operator operating the operation unit 33 differs from the subject of the blood pressure, the positional relationship between the subject and the information processing device 3 varies, and it is difficult to determine which orientation of the display screen 38 prevents the subject from viewing the display content of the display screen 38. However, in a case where the blood pressure monitor 2 is present at the rear surface of the information processing device 3 at least when the start operation is performed, the subject to which the blood pressure monitor 2 is attached is reliably present opposite to the display screen 38. For example, the sensor 35 such as the rear camera of the information processing device 3 is utilized to extract, from an image acquired by the rear camera, a logo of the product name of the blood pressure monitor 2, the model number of the blood pressure monitor 2, or the like, and depending on whether the logo or the model number corresponds to a particular or preset blood pressure monitor 2, whether the blood pressure monitor 2 is present opposite to the display screen 38 can be determined. In this way, by using, as an indicator, whether the blood pressure monitor 2 is present at the rear surface of the information processing device 3, the subject can be easily determined to be prevented from viewing the display content of the display screen 38.

Alternatively, for example, when the operation unit 33 receives the start operation, the control unit 37 may change the display on the display screen 38 of the display unit 34 to a display state in which the subject is prevented from viewing the display. The measurement start condition, used as a reference for the determination unit 41 of the control unit 37, may be determined to be satisfied when the display content of the display screen 38 changes to the display state in which the subject is prevented from viewing the display. For example, forcibly dimming the display screen 38 reliably prevents the subject from viewing the display content of the display screen 38. However, sudden erasure of the display screen may be mistaken for a failure or may surprise the subject, adversely affecting the blood pressure measurement. Thus, the display on the display screen 38 may be gradually dimmed, and in a case where the display has been dimmed to a certain degree, the measurement start condition may be determined to be satisfied. Alternatively, the control unit 37 may, for example, forcibly activate a screen saver when the operation unit 33 receives the start operation. In this way, by using, as an indicator, whether the display on the display screen 38 has changed to the state where the subject is prevented from viewing the display content of the display screen 38, the subject can be easily determined to be prevented from viewing the display content of the display screen 38.

Note that the notification unit 43 of the control unit 37 may, for example, display, on the display screen 38 of the display unit 34, a message prompting the blood pressure measurement with the blood pressure monitor 2, or may audibly output the message with the speaker 39 at a preset date and time or when the date and time is approached. Subsequently, the control unit 37 performs control to gradually dim the display on the display screen 38 of the display unit 34. Then, in a case where communication with the blood pressure monitor 2 is established within a certain period of time while the display is gradually dimmed, the display of the display screen 38 is recovered to the original state and determination of whether the measurement start condition is satisfied is started. Thus, at or near the date and time of the start of the measurement, the message prompting the blood pressure measurement on the blood pressure monitor is displayed or audibly outputted so as to prompt the subject to measure blood pressure at a preset date and time, and prevent a measurement forget. Moreover, when the display is gradually dimmed after the message prompting the blood pressure measurement is output, even in a case where the subject is operating any other application program on the information processing device 3, the operation is made difficult, allowing the subject to be diverted to the blood pressure measurement.

For example, when the measurement start condition is no longer satisfied after the connection unit 32 transmits the measurement start command to the blood pressure monitor 2, the control unit 37 may transmit a measurement stop command to the blood pressure monitor 2 through the connection unit 32. When, for example, the measurement stop command is received from the information processing device 3 through the blood pressure monitor connection unit 26, for example, the blood pressure monitor control unit 27 discontinues the measurement of blood pressure and causes a fluid in the fluid bag 24 to be discharged in a case where a compressed state by the fluid bag 24 provided in the cuff 22 of the blood pressure measurement unit 23 continues for a certain period of time or longer. In other words, for example, in the sense of a switch for emergency discharge (stop) in preparation for a case where an abnormality occurs in the fluid feeding mechanism of the blood pressure monitor 2 and the compressed state by the fluid bag 24 continues, “in a case where the compressed state continues for longer than a certain period of time and the information processing device 3 in, for example, a face-down state is suddenly moved,” processing for emergency stop can be executed. In this way, when the measurement start condition is no longer satisfied, the blood pressure measurement is discontinued and the fluid in the fluid bag 24 is discharged in a case where the compressed state by the fluid bag 24 of the cuff 22 continues for a certain period of time or longer. Thus, even in a case where the operation member is separate from the blood pressure monitor 2, the safety can be ensured.

In a case where the blood pressure monitor 2 further includes the emergency stop button 28 dedicated to emergency stop or also used as another button, in response to an operation of the emergency stop button, the blood pressure monitor control unit 27, for example, discontinues the measurement of the blood pressure and causes the fluid in the fluid bag 24 contained in the cuff 22 of the blood pressure measurement unit 23 to be discharged. In this way, in response to the operation of the emergency stop button 28 provided in the blood pressure monitor 2, the blood pressure monitor 2 discontinues the blood pressure measurement and causes the fluid in the fluid bag 24 to be discharged, ensuring safety.

### Effect of Application Example

As described above, in the biometric data measurement system 1 according to the application example, the blood pressure monitor 2 is caused to start the blood pressure measurement on condition that the subjected is prevented from viewing the display content of the display screen 38 of the display unit 34. Consequently, regardless of whatever content is caused to be displayed on the display unit 34 by an application program executed by the information processing device 3 in the background during the blood pressure measurement with the blood pressure monitor 2, the subject is prevented from viewing the content. This prevents the subject from being upset by the display content or being subjected to a rise in blood pressure due to the display content. Consequently, even in a case where the information processing device 3 is used as a user interface for the blood pressure monitor 2, accurate blood pressure measurements can be performed.

Note that the application example has been described in which, as an example, the biometric data measurement device is the blood pressure monitor 2 but that the biometric data measurement device is not limited to a blood pressure monitor and may of course be a device measuring other biometric data such as an electrocardiographic measurement device measuring electrocardiographic data.

Embodiments will be described below. Note that, in the following embodiments, the case in which the biometric data measurement device is a blood pressure monitor will be described as an example as is the case with the application example but that the biometric data measurement device is not limited to the blood pressure monitor and may be a device measuring other biometric data.

### <1> First Embodiment

Now, a first embodiment will be described.

### <1-1> Configuration

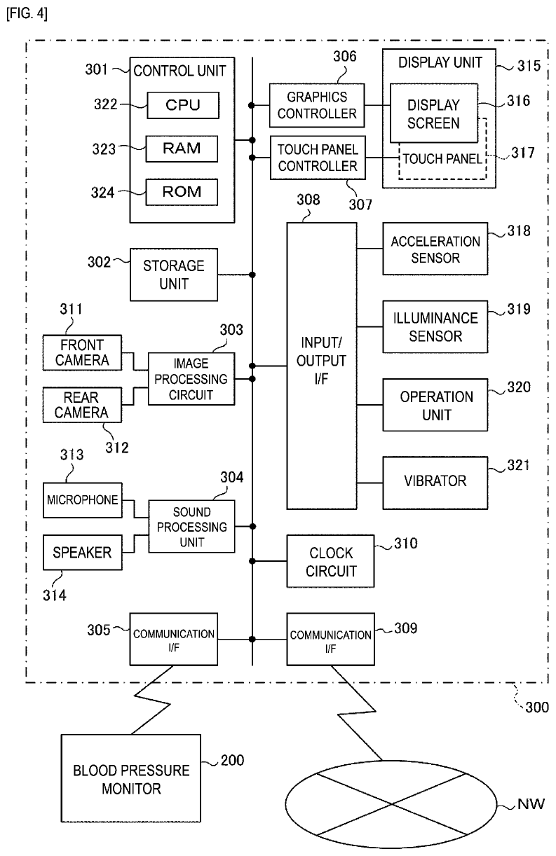

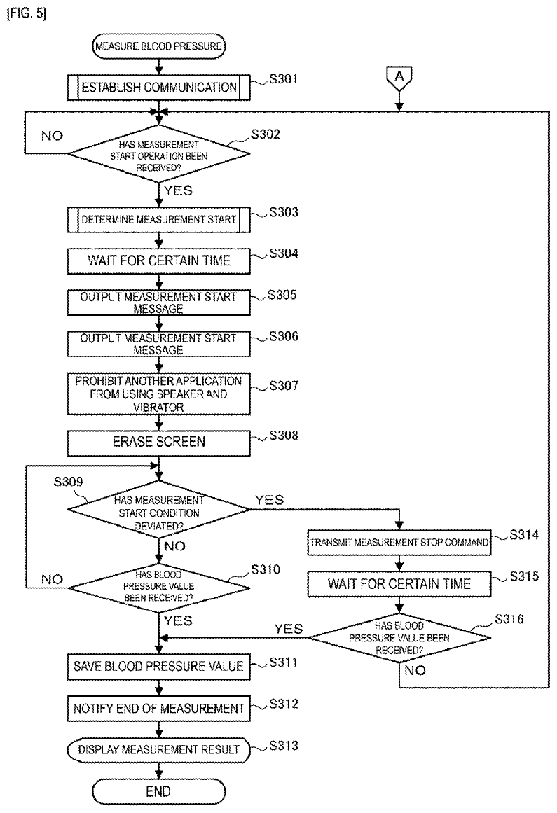

A configuration example of a biometric data measurement system according to the first embodiment will be described using FIGS. 3 and 4. As described above in the application example, the biometric data measurement system according to the first embodiment includes a blood pressure monitor 200 that measures the blood pressure of the subject, which is an example of the biometric data measurement device, and an information processing device 300 that controls a blood pressure measurement operation by the blood pressure monitor 200. FIG. 3 is a block diagram illustrating an example of a configuration of the blood pressure monitor 200 in the biometric data measurement system according to the first embodiment. FIG. 4 is a block diagram illustrating an example of a configuration of the information processing device 300 in the biometric data measurement system according to the first embodiment.

In the example of FIG. 3, the blood pressure monitor 200 is, for example, an electronic blood pressure monitor including a blood pressure monitor body 201 and a cuff 202 wrapped around a measured site such as the upper arm or wrist of the subject.

The cuff 202 corresponds to the cuff 22 as described above in the application example, and for example contains a fluid bag 203 corresponding to the fluid bag 24 as described above in the application example.

The blood pressure monitor body 201 corresponds to the blood pressure monitor body 21 as described above in the application example, and includes, for example, a control unit 204, an oscillation circuit 205 connected to the control unit 204 via a bus, a pump drive circuit 206, a valve drive circuit 207, an input/output interface 208, and a communication interface 209. Furthermore, the blood pressure monitor body 201 includes, for example, a pressure sensor 210 connected to the oscillation circuit 205, a piezoelectric pump 211 connected to the pump drive circuit 206, a valve 212 connected to the valve drive circuit 207, tubing 213 connecting the fluid bag 203 contained in the cuff 202 and a pressure sensor 210, a piezoelectric pump 211, and a valve 212, and a cuff wrapping state detection unit 214 and a power switch 215 connected to the input/output interface 208. Although not illustrated in particular, a power supply unit is provided that supplies power to each unit in the blood pressure monitor body 201. Note that, in FIG. 3, the input/output interface and the communication interface are described as “input/output I/F” and “communication I/F”, respectively.

The control unit 204 includes, for example, a CPU 216, a RAM 217, a ROM 218, and the like, and controls each component of the blood pressure monitor 200 according to the information processing. Instead of the CPU 216, any other processor such as an MPU may be used. The ROM 218 stores a program (e.g., a blood pressure measurement program) executed by CPU 216, for example. The RAM 217 is, for example, a memory used as a work memory by the CPU 216.

The CPU 216 may execute the required program to perform processing of the blood pressure monitor control unit 27 as described above in the application example. When the CPU 216 executes the required program, for example, the program to be executed which is stored in ROM 218 is developed into RAM 217. Then, the CPU 216 interprets and executes the program developed in the RAM 217 to control each component, for example.

The input/output interface 208 can, for example, input a detection signal from the cuff wrapping state detection unit 214 and an operation signal from the power switch 215 to the CPU 216.

The cuff wrapping state detection unit 214 corresponds to the detection unit 25 as described above in the application example, and detects the state of wrapping of the cuff 202 around the upper arm or wrist, corresponding to a measured site of the subject.

The cuff wrapping state detection unit 214 may detect the state of wrapping of the cuff 202 by detecting, for example, a state of electrical connection of an electrical contact provided in the cuff 202, marks aligned along the longitudinal direction of the cuff 202 and optically detected, a wrapping shape of the cuff 202 detected by a shape sensor such as a fiber sensor inserted through the cuff 202 in the longitudinal direction, and the like.

The power switch 215 is, for example, a switch turned on/off in response to the operation of a power button disposed on the surface of the blood pressure monitor body 201, and receives an instruction to turn on/off the power supply and an instruction for an emergency stop of the blood pressure measurement. The on/off instruction for the power supply and the instruction for an emergency stop of the blood pressure measurement can be distinguished, for example, by the on time of the power switch 215. For example, turn-on for at least a predetermined period of time such as two seconds, that is, long depression of the power button, corresponds to the on/off instruction for the power supply, and turn-on for shorter than two seconds, that is, short depression of the power button, corresponds to the instruction for an emergency stop of the blood pressure measurement. The operation signal indicating the on/off state of the power switch 215 is input to the CPU 216, for example, via the input/output interface 208. For example, based on the operation signal, the CPU 216 discriminates the on/off instruction for the power supply from the instruction for an emergency stop of the blood pressure measurement. Thus, the power switch 215 (power button) corresponds to the emergency stop button 28 as described above in the application example.

The communication interface 209 corresponds to the blood pressure monitor connection unit 26 as described in the above application example, and may be, but is not limited to, a near field wireless communication interface, for example, a BLE communication module. The communication interface 209 may be a wired communication interface, for example, a USB interface that communicates via a USB cable.

The following can constitute the blood pressure measurement unit 23 measuring the blood pressure of the subject as described above in the application example: the cuff 202 containing the fluid bag 203, and the pressure sensor 210, the piezoelectric pump 211, the valve 212, the tubing 213, the oscillation circuit 205, the pump drive circuit 206, and the valve drive circuit 207 in the blood pressure monitor body 201.

The pressure sensor 210 may be, for example, a piezoresistive pressure sensor. The oscillation circuit 205 converts an output from the pressure sensor 210 into a frequency, for example. The piezoelectric pump 211 is, for example, a pump that feeds air as a fluid to the fluid bag 203. The pump drive circuit 206 drives the piezoelectric pump 211, for example. The valve 212 is a valve that is opened and closed to adjust, for example, the pressure on a discharge side of the piezoelectric pump 211, i.e., the pressure in the fluid bag 203 (hereinafter referred to as the back pressure). The valve drive circuit 207 drives the valve 212, for example. The tubing 213 connects, for example, the pressure sensor 210, the piezoelectric pump 211, the valve 212, and the fluid bag 203 contained in the cuff 202, for example, and allow air as a fluid to circulate among the pressure sensor 210, the piezoelectric pump 211, the valve 212, and the fluid bag 203 contained in the cuff 202.

For example, in accordance with a program for controlling the blood pressure monitor 200 stored in the ROM 218, the CPU 216 performs control to drive the piezoelectric pump 211 via the pump drive circuit 206 and to drive the valve 212 via the valve drive circuit 207 in response to reception of a measurement start command from the information processing device 300 through the communication interface 209.

The piezoelectric pump 211 feeds air as a fluid to the fluid bag 203, for example, to increase the pressure (back pressure) in the fluid bag 203 contained in the cuff 202. The pump drive circuit 206, for example, drives the piezoelectric pump 211 based on a control signal provided by the CPU 216. The valve drive circuit 207, for example, opens and closes the valve 212 based on a control signal provided by the CPU 216. The valve 212 is opened and closed to control the back pressure, for example, by discharging air from the fluid bag 203 or sealing air in the fluid bag 203.

The pressure sensor 210 and the oscillation circuit 205 operate as pressure detection units that detect back pressure, for example. The pressure sensor 210 is, for example, a piezoresistive pressure sensor connected via the tubing 213 to the piezoelectric pump 211, the valve 212, and the fluid bag 203 contained in the cuff 202. In this example, the oscillation circuit 205, for example, oscillates based on an electrical signal from the pressure sensor 210 based on a change in electric resistance due to a piezoresistive effect and outputs, to the CPU 216, a frequency signal having a frequency corresponding to an electrical signal value from the pressure sensor 210.

The CPU 216, for example, calculates the current pressure of the fluid bag 203 based on the frequency signal input from the oscillation circuit 205. The CPU 216, for example, performs control to calculate a blood pressure value based on the pressure of the fluid bag 203, and to transmit the blood pressure value calculated to the information processing device 300 through the communication interface 209.

In the example illustrated in FIG. 4, the information processing device 300 includes, for example, a control unit 301, and components connected to the control unit 301 via a bus and including a storage unit 302, an image processing circuit 303, and a sound processing unit 304, a communication interface 305, a graphics controller 306, a touch panel controller 307, an input/output interface 308, a wireless communication interface 309, and a clock circuit 310. For example, a front camera 311 and a rear camera 312 are connected to the image processing circuit 303. For example, a microphone 313 and a speaker 314 are connected to the sound processing unit 304. For example, a display unit 315 is connected to the graphics controller 306 and the touch panel controller 307. The display unit 315 includes, for example, a display screen 316 and a touch panel 317. The display screen 316 is, for example, connected to the graphics controller 306, and the touch panel 317 is, for example, connected to the touch panel controller 307. The input/output interface 308 connects to, for example, an acceleration sensor 318, an illuminance sensor 319, an operation member 320, and a vibrator 321. Note that, in FIG. 4, the input/output interface and the communication interface are described as “input/output I/F” and “communication I/F”, respectively.

The control unit 301 includes, for example, a CPU 322, a RAM 323, a ROM 324, and the like, and controls each component according to information processing. Instead of the CPU 322, any other processor such as an MPU may be used. The ROM 324, for example, stores an operating system and any other program executed by the CPU 322. The RAM 323 is, for example, a memory used as a work memory by the CPU 322.

The CPU 322, for example, executes the required program to perform processing of the control unit 37, for example, the determination unit 41, the operation guidance unit 42, the notification unit 43, the inquiry unit 44, and the like as described above in the application example. The program may be, for example, stored in the storage unit 302 instead of the ROM 324. When the CPU 322 executes the required program, for example, the program to be executed that is stored in the ROM 324 and/or the storage unit 302 is developed into the RAM 323. Then, the CPU 322 interprets and executes the program developed in the RAM 323, and controls each component.

The storage unit 302 can store, for example, programs executed by the control unit 301 (e.g., an email application program, a messaging application program, and a blood pressure measurement application program), setting data needed to execute the program (e.g., data such as a mail server for emails and passwords), user specific information using information processing device 300, and so on. A storage medium included in the storage unit 302 may be any medium that accumulates information such as the programs by an electrical, magnetic, optical, mechanical, or chemical action such that a computer, a machine, and the like can read out the information such as the programs recorded. For example, an Electrically Erasable Programmable Read-Only Memory (EEPROM, trade name) or the like can be used as the storage medium included in the storage unit 302. Note that the storage unit 302 may be used as a work memory by the CPU 322.

For example, under the control of the CPU 322, the image processing circuit 303 performs predetermined image processing such as resizing, correction processing, filtering, or the like, on the image acquired by the front camera 311 and the rear camera 312. Here, the front camera 311 corresponds to the front camera 35A as described above in the application example, and the rear camera 312 corresponds to the rear camera as described above in the application example. For example, when the current camera image is displayed on the display screen 316 of the display unit 315 for composition during camera image capturing, the image processing circuit 303 resizes the images acquired by the front camera 311 or the rear camera 312 to the size (resolution) of the display screen 316, and stores the images in the RAM 323. When a certain image effect is applied by the camera application program, the CPU 322 may perform, for example, image processing corresponding to the effect on the image stored in the RAM 323, and display the image on the display screen 316. The image processing circuit 303 may have the function for image processing. For example, in response to a release operation by the operator, the image processing circuit 303 converts the image acquired by the front camera 311 or the rear camera 312 into an image of the set resolution, and stores the image in the storage unit 302. At this time, as in the case of display on the display screen 316, image processing can be executed on the image stored in the storage unit 302.

The sound processing unit 304 performs, for example, sound processing such as digital conversion, noise removal, or echo cancellation on a voice signal input from the microphone 313, and outputs the processed signal to the CPU 322. Under the control of the sound processing units 304 and the CPU 322, the voice signal generated by performing voice processing such as voice synthesis is output to the speaker 314. Here, the microphone 313 corresponds to the microphone as described above in the application example, and the speaker 314 corresponds to the speaker 39 of the notification unit 36 as described above in the application example.

The communication interface 305 corresponds to the connection unit 32 as described above in the application example, and is in near field wireless communication with, for example, the communication interface 209 of the blood pressure monitor 200. For example, the communication interface 305 may be, but is not limited to, a BLE communication module. For example, the communication interface 305 may be a wired communication interface such as a USB interface that communicates via a USB cable.

The graphics controller 306 is, for example, a display controller that controls the display screen 316 of the display unit 315. The display unit 315 corresponds to the display unit 34 as described in the application example. The display screen 316 corresponds to the display screen 38 as described above in the application example, and may be, for example, a liquid crystal display or an organic electroluminescent display.

The touch panel controller 307, for example, controls the touch panel 317, corresponding to a coordinate detection device disposed on the display screen 316. The touch panel controller 307, for example, acquires, from the touch panel 317, coordinate data indicating a touch position on the display screen 316 touched by the operator, and outputs the acquired coordinate data to the CPU 322.

The input/output interface 308 can, for example, input, to the CPU 322, a detection signal from the acceleration sensor 318 or the illuminance sensor 319, an operation signal from the operation member 320, and the like. The input/output interface 308 can, for example, output a vibration instruction from the CPU 322 to the vibrator 321.

The acceleration sensor 318 corresponds to the acceleration sensor as described above in the application example, and for example, detects the acceleration of the information processing device 300. The acceleration sensor 318 is, for example, not limited to detection of the accelerations in the directions of the X, Y, and Z axes, and may detect the acceleration in the rotational direction around each axis. The acceleration sensor 318 outputs an acceleration signal indicating the accelerations in the directions of the three axes and the acceleration in the rotational direction around each axis to the CPU 322 via the input/output interface 308. Note that instead of or in addition to the acceleration sensor 318, the information processing device 300 may include, for example, a gyroscopic sensor. The gyroscopic sensor corresponds to the gyroscopic sensor as described above in the application example, and can detect the angle of rotation when the information processing device 300 rotates around each of the X, Y, and Z axes, and output a rotation angle signal indicating the angle of rotation around each of the axes.

The illuminance sensor 319 corresponds to the illuminance sensor 35B as described above in the application example. The illuminance sensor 319 is, for example, a photodiode and detects the ambient brightness of the information processing device 300. In the present embodiment, the illuminance sensor 319 is disposed at a position near the display screen 316 on the front surface of the information processing device 300, and outputs a brightness signal indicating the ambient brightness of the display screen 316 to the CPU 322 via the input/output interface 308. Generally, the illuminance sensor 319 is utilized to adjust the brightness (luminance) of the display screen 316 in accordance with the ambient brightness.

Note that instead of or in addition to the illuminance sensor 319, the information processing device 300 may include, for example, a proximity sensor. The proximity sensor is a sensor that detects, in a non-contact manner, that an object to be detected has approached, and the proximity sensor is typically utilized to prevent the touch panel 317 from functioning when a phone call is made. Specifically, known examples of the proximity sensor include a high-frequency oscillating type utilizing electromagnetic induction, a magnetic type using a magnet, and a capacitive type that detects a change in capacitance between the object to be detected and the sensor, and the like. When detecting that the object to be detected has approached, the proximity sensor can output a proximity signal indicating that the object to be detected has approached.

The operation member 320 includes a switch that detects the operation of various operation buttons including the operation button 33A, for example, as described above in the application example. The operation member 320 can detect the operation of the operation button by the operator, and output an operation signal indicating the content of the operation to the CPU 322 via the input/output interface 308. The operation member 320, the touch panel 317 disposed on the display screen 316, and the touch panel controller 307 that controls the touch panel 317 correspond to the operation unit 33 as described above in the application example.

The vibrator 321 corresponds to the vibration member 40 of the notification unit 36 as described above in the application example. The vibrator 321 produces vibration in response to a vibration instruction provided by the CPU 322 via the input/output interface 308.

The wireless communication interface 309 corresponds to a communication unit 31 as described above in the application example, and may include, for example, a Wi-Fi module. The wireless communication interface 309 is, for example, connected to the communication network NW such as the Internet or a telephone network via a Wi-Fi base station not illustrated, and can communicate with an external device via the communication network NW.

The clock circuit 310 includes, for example, a clock, and can acquire the current date and time.

### <1-2> Operation

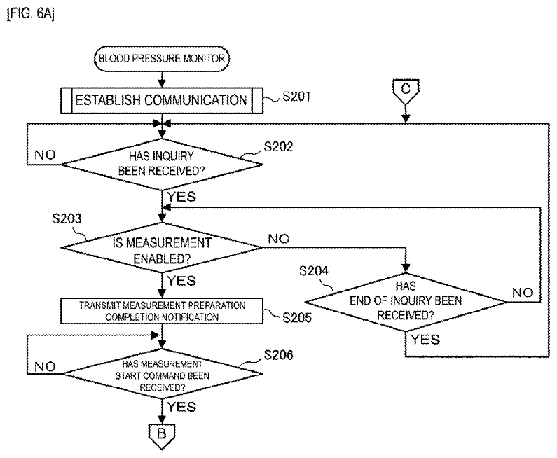

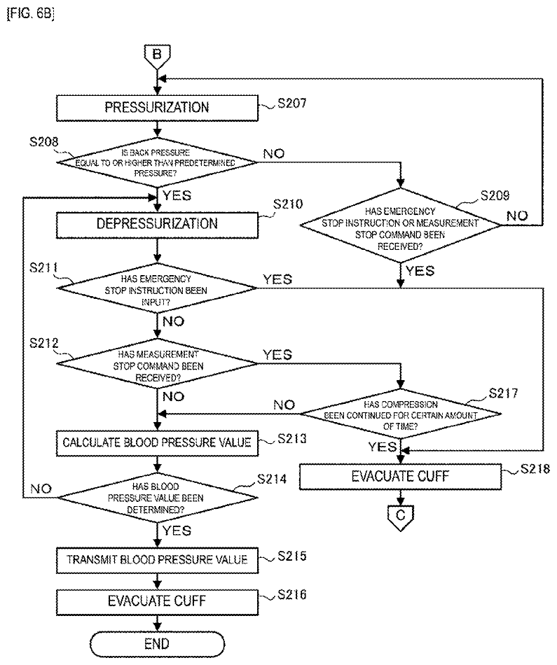

Now, an operation example of the biometric data measurement system according to the first embodiment will be described using FIGS. 5 and 6.

FIG. 5 is a flowchart illustrating an example of a processing procedure for the information processing device 300 in accordance with the blood pressure measurement application program. The processing procedure is started, for example, when the touch panel 317 is operated at a position corresponding to the icon of the blood pressure measurement application program displayed on the display screen 316, and the CPU 322 determines that activation of the blood pressure measurement application program has been instructed. FIG. 6 is a flowchart illustrating an example of a processing procedure for the blood pressure monitor 200. The processing procedure is started, for example, when the CPU 216 determines that power-on has been instructed based on an operation signal from the power switch 215 in response to the operation, by the subject, of the power button disposed on the front surface of the blood pressure monitor body 201.

Note that the processing procedure described below is merely an example, and each process may be changed to the extent possible. Further, in the processing procedure described below, steps can be omitted, substituted, and added in accordance with the embodiment as appropriate.

### <1-2-1> Communication Establishment Operation

### [Step S301]

First, the CPU 322 of the information processing device 300 establishes near field communication with the blood pressure monitor 200 through the communication interface 305.

### [Step S201]

The CPU 216 of the blood pressure monitor 200 similarly establishes a near field communication with the information processing device 300 through the communication interface 209.

### <1-2-2> Measurement Start Determination Operation

### [Step S302]

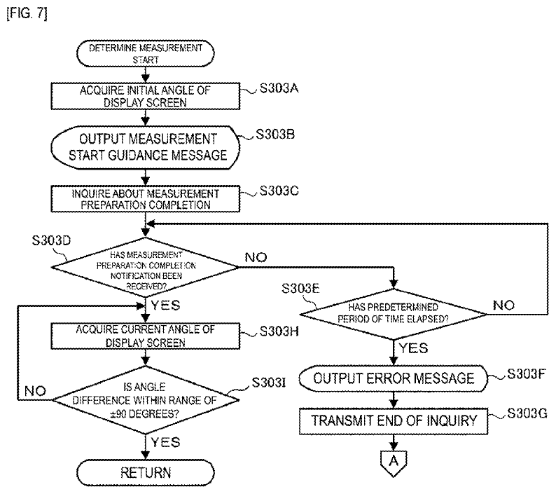

The CPU 322 of the information processing device 300 waits for the operator to perform the start operation for the blood pressure measurement after establishing near field communication with the blood pressure monitor 200. In other words, the CPU 322 causes the graphics controller 306 to display, on the display screen 316 of display unit 315, an initial screen for the blood pressure measurement application program including the icon 38A for instructing the start of the blood pressure measurement as described above in the application example. Then, the CPU 322 determines whether the position on the touch panel 317 corresponding to the icon 38A has touched, based on touch coordinates input from the touch panel controller 307. In a case where the blood pressure measurement start operation is determined not to have been performed (step S302, NO), the CPU 322 repeats the processing in step S302. In a case where the blood pressure measurement start operation is determined to have been performed (step S302, YES), the CPU 322 advances the processing to step S303.

[Step S303] In a case that in step S302, the blood pressure measurement start operation is determined to have been performed (step S302, YES), the CPU 322 executes measurement start determination processing to instruct the blood pressure monitor 200 to start measurement when the measurement start condition is satisfied. FIG. 7 is a flowchart illustrating an example of a processing procedure for the information processing device.

### [Step S303A]

In the measurement start determination processing, the CPU 322 first acquires the initial angle of the display screen initial angle. For example, based on the detection signal from the acceleration sensor 318 acquired via the input/output interface 308, the CPU 322 can calculate the angle of the information processing device 300, that is, the display screen 316 with respect to the gravitational direction, in other words, the direction in which display screen 316 faces. The acquired initial angle of the display screen is stored in the RAM 323 or the storage unit 302.

### [Step S303B]

Then, the CPU 322 outputs, through the display screen 316 and/or the speaker 314, a measurement start guidance message prompting movement of the information processing device 300 to satisfy the measurement start condition. For example, the CPU 322 may can cause the graphics controller 306 to display the characters of the measurement starting guidance message on the display screen 316, and cause the sound processing unit 304 to emit the voice of the measurement start guidance message through the speaker 314. For example, the content of the measurement start guidance message is stored in the storage unit 302 in advance as a part of the blood pressure measurement application program. Thus, the CPU 322 can perform the processing of the operation guidance unit 42 as described above in the application example.

### [Step S303C]

Then, the CPU 322 transmits an inquiry for measurement preparation completion to the blood pressure monitor 200 through the communication interface 305. Thus, the CPU 322 may perform processing of the inquiry unit 44 as described above in the application example.

### [Step S303D]

Subsequently, the CPU 322 determines whether a measurement preparation completion notification has been received from the blood pressure monitor 200 through the communication interface 305. In a case where the measurement preparation completion notification is determined not to have been received (step S303D, NO), the CPU 322 advances the processing to step S303E. In a case where the measurement preparation completion notification is determined to have been received (step S303D, YES), the CPU 322 advances the processing to step S303H.

### [Step S303E]

In step S303D described above, in a case where the measurement preparation completion notification is determined not to have been received from the blood pressure monitor 200 (Step S303D, NO), the CPU 322 determines whether a predetermined period of time, for example, 5 seconds have elapsed since the transmission of the inquiry for measurement preparation completion. The 5 seconds are illustrative and no such limitation is intended. In a case where the predetermined period of time is determined not to have elapsed (step S303E, NO), the CPU 322 returns the processing to step S303D described above. In a case where the predetermined period of time is determined to have elapsed (step S303E, YES), the CPU 322 advances the processing to step S303F.

Thus, by repeating the processing in steps S303D and S303E, the CPU 322 waits for the predetermined period of time to receive the measurement preparation completion notification.

### [Step S202]

After establishing near field communication with the information processing device 300, the CPU 216 of the blood pressure monitor 200 waits to receive an inquiry for measurement preparation completion from the information processing device 300. In other words, the CPU 216 determines whether the inquiry for measurement preparation completion has been received from the information processing device 300 through the communication interface 209. In a case where the inquiry for measurement preparation completion is determined not to have been received (step S202, NO), the CPU 216 repeats the processing in step S202. In a case where the inquiry for measurement preparation completion is determined to have been received (step S202, YES), the CPU 216 advances the processing to step S203.

### [Step S203]

In step S202 described above, in a case where the inquiry for measurement preparation completion is determined to have been received from the information processing device 300 (step S202, YES), the CPU 216 determines whether the blood pressure measurement is enabled to be performed. For example, the CPU 216 can determine whether the measurement has been enabled or not, based on of the state of wrapping of the cuff 202 indicated by the detection signal acquired from the cuff wrapping state detection unit 214 via the input/output interface 208. In a case where the blood pressure measurement is determined not to have been enabled (step S203, NO), the CPU 216 advances the processing to step S204. In a case where the blood pressure measurement is determined to have been enabled (step S203, YES), the CPU 216 advances the processing to step S205.

### [Step S204]

In step S203 described above, in a case where the blood pressure measurement is determined not to have been enabled (step S203, NO), the CPU 216 causes the communication interface 209 to determine whether the end of the inquiry has been received from the information processing device 300. In a case where the end of the inquiry is determined not to have been received (step S204, NO), the CPU 216 returns the processing to step S203 described above.

### [Step S303F]

In step S303E described above, in a case where a predetermined period of time is determined to have elapsed with the measurement preparation completion notification not received from the blood pressure monitor 200 (step S303E, YES), the CPU 322 outputs an error message via the display screen 316 and/or the speaker 314. For example, the CPU 322 causes the graphics controller 306 to display the characters of an error message on the display screen 316, and causes the sound processing unit 304 to emit the voice of the error message through the speaker 314. For example, the content of the error message is stored in the storage unit 302 in advance as a part of the blood pressure measurement application program. The content of the error message may include a message indicating that the blood pressure monitor 200 is not ready, or an instruction message for an instruction to correctly re-wrap the cuff 202.

### [Step S303G]

Subsequently, the CPU 322 transmits the end of the inquiry to the blood pressure monitor 200 through the communication interface 305. Then, the measurement start determination process is ended, and the processing is returned to step S302.

### [Step S204]

In step S204 described above, in a case where the end of the inquiry is determined to have been received (step S204, YES), the CPU 216 of the blood pressure monitor 200 returns the processing to step S202 above. In other words, when the preparation is not complete even after the predetermined period of time has elapsed since the inquiry for the measurement preparation completion, the information processing device 300 outputs an error message, and in response, the subject re-wraps the cuff 202 and performs the measurement start operation again using the information processing device 300. Thus, the CPU 216 returns the processing back to step S202 described above and waits to receive an inquiry for measurement preparation completion from the information processing device 300.

### [Step S205]

In contrast, in step S203 described above, in a case where the blood pressure measurement is determined to have been enabled (step S203, YES), the CPU 216 transmits a measurement preparation completion notification to the information processing device 300 through the communication interface 209.

### [Step S303H]

In step S303D described above, in a case where the measurement preparation completion notification is determined to have been received from the blood pressure monitor 200 (step S303D, YES), the CPU 322 of the information processing device 300 acquires the current angle of the display screen 316. For example, based on the detection signal from the acceleration sensor 318 acquired via input/output interface 308, the CPU 322 calculates the angle of the information processing device 300, that is, the display screen 316 with respect to the gravitational direction, in other words, the direction in which display screen 316 faces. The acquired current angle of the display screen is stored in the RAM 323 or the storage unit 302.

### [Step S303I]

Then, the CPU 322 determines whether the difference between the initial angle and the current angle of the display screen 316 stored in the RAM 323 or the storage unit 302 is outside the range of the predetermined angle, for example, the range of ±90 degrees. The ±90 degree are illustrative and no such limitation is intended. The predetermined angle is stored in the storage unit 302 in advance as a part of the blood pressure measurement application program. As described above in the application example, in a case where, for example, the angle difference exceeds the range of ±90 degrees, the angle may be determined to equal a measurement start angle at which the subject is prevented from viewing the display screen. However, this difference in angle is with respect to one of the x-axis corresponding to the left-right direction (lateral direction) of the display screen 316 or the y-axis corresponding to the vertical direction (longitudinal direction), and the angle related to the z-axis corresponding to the front-back direction (depth direction) is not included in this determination. In a case where the angle difference is determined to not to be outside the range of ±90 degrees (step S303I, NO), the CPU 322 returns the processing to step S303H described above. In a case where the angle difference is determined to be outside the range of ±90 degrees (step S303I, YES), the CPU 322 determines that the measurement start condition is satisfied to end the measurement start determination processing and advances the processing proceeds to step S304. In this way, the CPU 322 can execute the processing of the determination unit 41 in which the measurement start condition is determined to be satisfied when the angle of the display screen 38 equals the measurement start angle at which the subject is prevented from viewing the display screen 38, as described above in the application example.

### [Step S304]

As described above, in a case where the measurement start condition is satisfied, the CPU 322 waits for a certain period of time, e.g., 3 seconds. The 3 seconds are illustrative and no such limitation is intended.

### [Step S305]

Subsequently, the CPU 322 outputs a measurement start message through the speaker 314. In other words, the CPU 322 emits the voice of the measurement starting message from the speaker 314 through the sound processing unit 304. For example, the content of the measurement start message is stored in the storage unit 302 in advance as a part of the blood pressure measurement application program. Note that in this case, because the subject is prevented from viewing the display content of the display screen 316, the CPU 322 does not display the measurement start message on the display screen 316 unlike in the case of other messages.

### [Step S306]

Subsequently, the CPU 322 transmits a measurement start command to the blood pressure monitor 200 via the communication interface 305.

In this manner, the transmission of the measurement start command is performed after a wait for a certain period of time, rather than immediately after the output of the measurement start message to the blood pressure monitor 200. Thus, as is the case with common blood pressure monitors, the subject can behave from sensing of the start of the measurement.

### [Step S307]

The CPU 322 prohibits the speaker 314 and the vibrator 321 from being used by the application program running in the background. For example, the CPU 322 prevents a ringtone (ringer melody) for the reception of an email or a message in the email application program or messaging application program from being emitted from the speaker 314.

Note that for a ringtone (ringer melody) for reception of a telephone call, the use of the speaker 314 is not prohibited. Unlike emails and messages, telephone calls are often used in more urgent cases and thus needs to have priority over blood pressure measurement. Some messaging application programs include a telephone call function via the Internet, and a ringtone related to telephone calls in such an application program is desirably handled similarly to incoming calls.

### [Step S308]

Furthermore, the CPU 322 erases the display screen 316 by, for example, stopping the supply of power from a power supply unit (not illustrated) to the display screen 316. Erasing the display screen 316 enables suppression of the power consumption of the information processing device 3.

Note that the processing in steps S307 and S308 may be performed before step S306 described above or during the wait in step S304 described above. Either of the processing in step S307 and the processing in step S308 may be performed before the other. Furthermore, the processing in step S307 and/or step S308 may be omitted.

### [Step S206]

After transmitting the measurement preparation completion notification to the information processing device 300 in step S205 described above, the CPU 216 of the blood pressure monitor 200 waits to receive the measurement start command from the information processing device 300. In other words, the CPU 216 determines whether or not the measurement start command has been received from the information processing device 300 through the communication interface 209. In a case where the measurement start command is determined not to have been received (step S206, NO), the CPU 216 repeats the processing in step S206. In a case where the measurement start command is determined to have been received (step S206, YES), the CPU 216 advances the processing to step S207.

### <1-2-3> Blood Pressure Measurement Operation

### [Step S207]

In a case where in step S206 described above, the measurement start command is determined to have been received from the information processing device 300 (step S206, YES), the CPU 216 introduces a fluid (air) into the fluid bag 203 contained in the cuff 202 to increase the pressure (back pressure) in the fluid bag 203. For example, the CPU 216 first closes the valve 212 via the valve drive circuit 207 and then drives the piezoelectric pump 211 via the pump drive circuit 206 to feed air to the fluid bag 203. This inflates the fluid bag 203 while gradually increasing the back pressure.

### [Step S208]

Then, the CPU 216 determines whether the back pressure detected by the pressure sensor 210 is equal to or higher than a predetermined pressure based on a frequency signal input from the oscillation circuit 205. The predetermined pressure is a pressure sufficiently higher than the systolic blood pressure of the subject (e.g. the systolic blood pressure+30 mmHg). This may be pre-stored in the ROM 218 or any other memory not illustrated, or the CPU 216 may estimate and determine the systolic blood pressure during pressurization in accordance with a predetermined calculation formula (see, e.g., JP 2001-70263). In a case where the back pressure is not equal to or higher than the predetermined pressure (step S208, NO), the CPU 216 advances the processing to step S209. In a case where the back pressure is equal to or higher than the predetermined pressure (step S208, YES), the CPU 216 advances the processing to step S210.

### [Step S209]

In a case where in step S208 described above, that the back pressure is determined to be equal to or higher than the predetermined pressure (step S208, NO), the CPU 216 determines whether a blood pressure measurement emergency stop command has been input to the blood pressure monitor 200 or not or whether a measurement stop command has been received from the information processing device 300 or not. For example, the CPU 216 can determine whether the blood pressure measurement emergency stop instruction has been input by short depression of the power button, based on an operation signal from the power switch 215 in response to the subject operation of the power button disposed on the front surface of the blood pressure monitor body 201. For example, the CPU 216 can determine whether the measurement stop command has been received or not based on the result of reception by the communication interface 209 that communicates with the information processing device 300. In a case where the input of the blood pressure measurement emergency stop instruction or the reception of the measurement stop command is determined not to have been performed (step S209, NO), the CPU 216 returns the processing to step S207 described above and continues pressurization. In a case where the input of the blood pressure measurement emergency stop instruction or the reception of the measurement stop command is determined to have been performed (step S209, YES), the CPU 216 advances the processing to step S218.

### [Step S210]

In step S208 described above, in a case where the back pressure is determined to be equal to or higher than the predetermined pressure (step S208, YES), the CPU 216 deflates the fluid bag 203 and gradually reduces the back pressure. For example, the CPU 216 stops the piezoelectric pump 211 via the pump drive circuit 206 and then performs control to progressively open the valve 212 via the valve drive circuit 207. For a depressurization rate in this case, for example, a target depressurization rate corresponding to a target during pressurization of the cuff 202 is set, and the CPU 216 controls a degree of opening of the valve 212 to achieve the target depressurization rate.

### [Step S211]

The CPU 216 determines whether the blood pressure measurement emergency stop instruction has been input. For example, the CPU 216 can determine whether the blood pressure measurement emergency stop instruction has been input by short depression of the power button, based on an operation signal from the power switch 215 in response to the subject operation of the power button disposed on the front surface of the blood pressure monitor body 201. In a case where the blood pressure measurement emergency stop instruction is determined not to have been input (step S211, NO), the CPU 216 advances the processing to step S212. In a case where the blood pressure measurement emergency stop instruction is determined to have been input (step S211, YES), the CPU 216 advances the processing to step S218.

### [Step S212]

In step S211 described above, in a case where the blood pressure measurement emergency stop instruction is determined not to have been input (step S211, NO), the CPU 216 determines whether the measurement stop command has been received from the information processing device 300. For example, the CPU 216 can determine whether the measurement stop command has been received or not based on the result of reception by the communication interface 209 that communicates with the information processing device 300. In a case where the measurement stop command is determined not to have been received (step S212, NO), the CPU 216 advances the processing to step S213. In a case where the measurement stop command is determined to have been received (step S212, YES), the CPU 216 advances the processing to step S217.

### [Step S213]

In a case where in step S212 described above, the measurement stop command is determined not to have been received (step S212, NO), the CPU 216 calculates the blood pressure value. For example, in the depressurization process described above, the pressure sensor 210 detects, via the cuff 202, a back pressure signal representing the pressure of the cuff 202. Based on the back pressure signal, the CPU 216 can calculate the blood pressure value (systolic blood pressure and diastolic blood pressure) according to the oscillometric method. Note that the calculation of the blood pressure value is not limited to the depressurization process and may be performed during the pressurization process.

### [Step S214]

The CPU 216 determines whether the blood pressure value has been determined or not. For example, in a case where both systolic blood pressure and diastolic blood pressure have been calculated, the blood pressure value is determined to have been determined. In a case where the blood pressure value is determined not to have been determined (step S214, NO), the CPU 216 returns the processing to step S210 described above to continue depressurization. In a case where the blood pressure value is determined to have been determined (step S214, YES), the CPU 216 advances the processing to step S215.

### [Step S215]

In a case where in step S214 described above, the blood pressure value has been determined (step S214, YES), the CPU 216 transmits the blood pressure value calculated to the information processing device 300 through the communication interface 209.

### [Step S216]

Subsequently, the CPU 216 opens the valve 212 via the valve drive circuit 207 to evacuate air in the fluid bag 203 of the cuff 202.

### [Step S309]

After erasing the display screen 316 in step S308 described above, the CPU 322 of the information processing device 300 determines whether the measurement start condition has deviated or not. As described above, the measurement start condition is, for example, that the current angle of the display screen 316 exceeds a predetermined angle from the initial angle, for example, the range of ±90 degrees. Thus, in step S309, whether the difference between the initial angle and the current angle of the display screen 316 is within a predetermined angle, for example within ±90 degrees. In a case where the measurement start condition is determined not to have deviated (step S309, NO), the CPU 322 advances the processing to step S310. In a case where the measurement start condition is determined to have deviated (step S309, YES), the CPU 322 advances the processing to step S314.

### [Step S310]

In step S309 described above, in a case where the measurement start condition is determined not to have deviated (step S309, NO), the CPU 322 determines whether the blood pressure value has been received from the blood pressure monitor 200 through the communication interface 305 or not. In a case where the blood pressure value is determined not to have been received (step S310, NO), the CPU 322 returns the processing to step S309 described above. In a case where the blood pressure value is determined to have been received (step S310, YES), the CPU 322 advances the processing to step S311.

By repeating step S309 and step S310, the CPU 322 waits for the blood pressure monitor 200 to measure the blood pressure value.

### [Step S311]

In a case where in step S310 described above, the blood pressure value is determined to have been received (step S310, YES), the CPU 322 stores the received blood pressure value in the storage unit 302.

### [Step S312]

Subsequently, the CPU 322 notifies the end of the measurement. For example, the CPU 322 causes the sound processing unit 304 to emit the voice of a measurement end message from the speaker 314. For example, the content of the measurement end message is stored in the storage unit 302 in advance as a part of the blood pressure measurement application program. In this case, the CPU 322 may further cause the vibrator 321 to vibrate via the input/output interface 308.

[Step S313]

Then, the CPU 322 displays the measurement result stored in the storage unit 302 on the display screen 316 via the graphics controller 306. By making the display screen 316 of the information processing device 300 visible in response to the notification of the end of measurement in step S312 described above, the subject can view the measurement result displayed on the display screen 316. Note that the measurement result displayed on the display screen 316 may include only the currently measured blood pressure value or a previously measured blood pressure value and the current blood pressure value presented together. Furthermore, processed information may be presented such as temporal changes in blood pressure value, statistics, or the like. What measurement result is displayed depends on the blood pressure measurement application program and selection by the operator.

### <1-2-4> Emergency Stop Operation

[Step S209, step S211]

The CPU 216 of the blood pressure monitor 200 determines, in step S209 and step S211 described above, whether a blood pressure measurement emergency stop instruction has been input as described above. For example, in a case where the blood pressure measurement emergency stop instruction is determined to have been input by the subject by short-time turn-on of the power switch 215 based on short depression of the power button disposed on the front surface of the blood pressure monitor body 201 (step S209 or step S211, YES), the CPU 216 advances the processing to step S218.

### [Step S218]

In a case where in step S209 or step S211 described above, the blood pressure measurement emergency stop instruction is determined to have been input by the subject (step S209 or step S211, YES), the CPU 216 opens the valve 212 via the valve drive circuit 207 and evacuates air in the fluid bag 203 of the cuff 202. The CPU 216 then returns the processing to step S202 described above.

A situation where a power button functioning as an emergency stop button for the blood pressure monitor 200 is operated (when the power switch 215 is turned on for a short time) is assumed to be a case where the subject desires to immediately discontinue the blood pressure measurement. In particular, the operation of the emergency stop button during depressurization includes a case where an abnormality occurs in the piezoelectric pump 211 and an emergency situation may occur in which the pressure in the fluid bag 203 is prevented from being reduced. Thus, in response to the operation of the emergency stop button, the blood pressure monitor 200 immediately discontinues the blood pressure measurement and causes the fluid in the fluid bag 203 to be discharged. Thus, the safety of the blood pressure measurement can be ensured.

### [Step S309]

On the other hand, as described above, the CPU 322 of the information processing device 300 determines, in step S309 described above, whether the measurement start condition has deviated. For example, in a case where the difference between the initial angle and the current angle of the display screen 316 is within the range of a predetermined angle, for example the range of ±90 degrees, the CPU 322 determines that the measurement start condition has deviated. In a case where the measurement start condition is determined to have deviated (step S309, YES), the CPU 322 proceeds the processing to step S314.

### [Step S314]

In a case where in step S309 described above, the measurement start condition is determined to have deviated (step S309, YES), the CPU 322 transmits a measurement stop command to the blood pressure monitor 200 through the communication interface 305.

### [Step S315]

The CPU 322 then waits for a certain period of time, e.g., for the time required for normal blood pressure measurement.

### [Step S209]

The CPU 216 of the blood pressure monitor 200 determines, in step S209 described above, whether the measurement stop command has been received from the information processing device 300, as described above. For example, in a case where the measurement stop command is determined to have been received from the information processing device 300 based on the result of reception by the communication interface 209 that communicates with the information processing device 300 (step S209, YES), the CPU 216 advances the processing to step S218.

### [Step S218]

In step S209 above, in a case where the measurement stop command is determined to have been received from the information processing device 300 (step S209, YES), the CPU 216 opens the valve 212 via the valve drive circuit 207 and evacuates the air in the fluid bag 203 of the cuff 202.

### [Step S316]

After waiting for a certain period of time in step S315 described above, the CPU 322 of the information processing device 300 determines whether the blood pressure value has been received from the blood pressure monitor 200 through the communication interface 305 or not. In a case where the blood pressure value is determined not to have been received (step S316, NO), the CPU 322 returns the processing to step S302 described above.

In this way, when the measurement start condition deviates on the information processing device 300 side while the blood pressure monitor 200 is being pressurized, the pressure in the fluid bag 203 is not sufficient yet to enable the blood pressure to be measured using the blood pressure monitor 200. Thus, the blood pressure measurement is discontinued. In a case where the blood pressure measurement is performed again, the processing may be repeated starting at step S202 and at step S302.

### [Step S212]

As described above, the CPU 216 of the blood pressure monitor 200 determines, in step S212 described above, whether the measurement stop command has been received from the information processing device 300 or not. For example, in a case where the measurement stop command is determined to have been received from the information processing device 300 based on the result of reception by the communication interface 209 that communicates with the information processing device 300 (step S212, YES), the CPU 216 advances the processing to step S217.

### [Step S217]

In step S212 described above, in a case where the measurement stop command is determined to have been received from the information processing device 300 (step S212, YES), the CPU 216 determines whether compression at or above a predetermined pressure has continued for a certain period of time, for example, several seconds. For example, the CPU 216 makes this determination based on the back pressure detected by the pressure sensor 210 and indicated by a frequency signal input from the oscillation circuit 205. The several seconds are illustrative and no such limitation is intended. In a case where the compression is determined not to have continued for the certain period of time (step S217, NO), the CPU 216 advances the processing to step S213 described above. In a case where the compression is determined to have continued for the certain period of time (step S217, YES), the CPU 216 advances the processing to step S218 described above.

### [Step S218]

In step S217 described above, in a case where the compression is determined to have continued for the certain period of time (step S217, YES), the CPU 216 opens the valve 212 via the valve drive circuit 207 and evacuates the air in the fluid bag 203 of the cuff 202.

In this way, when the measurement start condition deviates on the information processing device 300 side, in a case where the compression has continued at the blood pressure monitor 200 for the certain period of time, the CPU 216 of the blood pressure monitor 200 determines that the act of the deviation has been intentionally carried out by the subject corresponding to the operator of the information processing device 300 instead of operating the emergency stop button of the blood pressure monitor 200. Thus, the CPU 216 immediately discontinues the blood pressure measurement and causes the fluid in the fluid bag 203 to be discharged. Thus, the safety of the blood pressure measurement can be ensured.

### [Steps S213 to S216]

In contrast, in step S217 described above, in a case where the compression is determined not to have continued for the certain period of time (step S217, NO), the CPU 216 advances the processing to step S213 and executes the processing in steps S213 to S216 described above. Specifically, once the blood pressure value is calculated and determined, the blood pressure value calculated is transmitted to the information processing device 300 through the communication interface 209, and the valve 212 is opened via the valve drive circuit 207 to evacuate the air in the fluid bag 203 of the cuff 202.

### [Step S316]

After waiting for a certain period of time in step S315 described above, the CPU 322 of the information processing device 300 determines whether the blood pressure value has been received from the blood pressure monitor 200 through the communication interface 305 or not. In a case where the blood pressure value is determined not to have been received (step S316, NO), the CPU 322 returns the processing to step S302 described above. In a case where that the blood pressure value has been received (step S316, YES), the CPU 322 advances the processing to step S311 above.

### [Steps S311 to S313]

In step S316 described above, in a case where the blood pressure value is determined to have been received (step S316, YES), the CPU 322 proceeds to step S311 described above, and executes the processing in steps S311 to S313 described above. In other words, after the blood pressure value received is saved in the storage unit 302, the end of the measurement is notified, and the measurement result is displayed on the display screen 316.

In this way, when the measurement start condition deviates on the information processing device 300 side, in a case where the compression has not continued at the blood pressure monitor 200 for the certain period of time, the CPU 216 of the blood pressure monitor 200 can determine that the act of the deviation has been inadvertently carried out by the subject corresponding to the operator of the information processing device 300, and continue the blood pressure measurement.

### <1-3> Effect

According to the first embodiment described above, the blood pressure monitor 200 is caused to start the blood pressure measurement on condition that the subject corresponding to the operator of the information processing device 300 is prevented from viewing the display content of the display screen 316 of the display unit 315. Thus, regardless of whatever content is caused to be displayed on the display screen 316 by an application program executed by the information processing device 300 in the background during the blood pressure measurement with the blood pressure monitor 200, the subject is prevented from viewing the content. This prevents the subject from being upset by the display content or being subjected to a rise in blood pressure due to the display content. Consequently, even in a case where the information processing device 300 is used as a user interface for the blood pressure monitor 200, accurate blood pressure measurement can be performed.

Note that, in the first embodiment, the measurement start condition is that the angle of the display screen 316 equals the measurement start angle at which the subject is prevented from viewing the display screen 316, and this state is determined by the difference between the initial angle and the current angle of the display screen 316. However, whether the angle of the display screen 316 equals the measurement start angle or not is not limited to this. For example, the determination may be made only by the current angle of the display screen 316, that is, the direction in which the display screen 316 faces. For example, the situation in which the subject measures the blood pressure while lying down is exceptional, and thus the display screen 316 is unlikely to face in the gravitational direction during the blood pressure measurement. Thus, the measurement start angle may correspond to the state where the display screen 316 is at the angle at which the display screen 316 approximately faces the gravitational direction.

### <1-4> Modified Examples

The measurement start conditions other than the measurement start angle will be described below as modified examples.

### 1-4-1

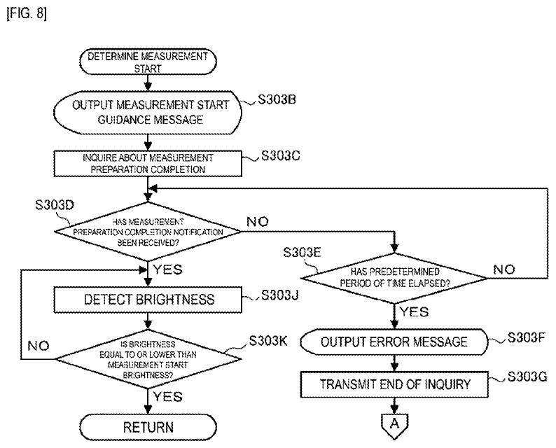

FIG. 8 is a flowchart illustrating an example of a processing procedure of measurement start determination processing in a first modified example of the first embodiment.

### [Steps S303B to S303G]

In the first modified example, when the measurement start determination processing is started, the CPU 322 executes the processing in steps S303B to S303G described above without acquiring the initial angle of the display screen as in step S303A of the first embodiment.

### [Step S303.1]

In step S303D described above, in a case where the measurement preparation completion notification is determined to have been received from the blood pressure monitor 200 (step S303D, YES), the CPU 322 detects the brightness of the display screen 316. For example, the CPU 322 detects the illuminance of the information processing device 300, that is, the display screen 316, in other words, brightness attributed to ambient light, based on the detection signal from the illuminance sensor 319 acquired via the input/output interface 308. Alternatively, the CPU 322 can acquire an image from the front camera 311 via the image processing circuit 303, and detect the brightness (illuminance) of the display screen 316 based on luminance information regarding the entire image. The brightness detected is stored in the RAM 323 or the storage unit 302.

### [Step S303K]

Then, the CPU 322 determines whether the brightness (illuminance) of the display screen 316 stored in the RAM 323 or the storage unit 302 is equal to or less than the predetermined measurement start brightness (illuminance) or not. The predetermined measurement start brightness (illuminance) is set to a brightness obtained when the display screen 316 is covered. This predetermined measurement start brightness (illuminance) is stored in the storage unit 302 in advance as a part of the blood pressure measurement application program. In a case where the brightness (illuminance) of the display screen 316 is not equal to or less than the predetermined measurement start brightness (illuminance) (step S303K, NO), the CPU 322 returns the processing to step S303J. In a case where the brightness (illuminance) of the display screen 316 is equal to or less than the predetermined measurement start brightness (illuminance) (step S303K, YES), the CPU 322 determines that the measurement start condition is satisfied, ends the measurement start determination process, and advances the processing to step S304. Thus, when the display screen 38 is covered, as described above in the application example, the CPU 322 can perform the processing of the determination unit 41 determining that the measurement start condition is satisfied.

In this way, the measurement start condition may be that the display screen 316 is covered. For example, in a case where the information processing device 300 is placed in a notebook type cover, closing the cover covers the display screen 316 to prevent the subject from viewing the display screen 316. When the information processing device 300 is laid face down on a desk or the like, the display screen 316 is covered by a top plate of the desk. In a case where the measurement start condition is that the display screen 316 is covered, prevention of the subject from viewing the display content of the display screen 316 can be easily determined.

### <1-4-2> Second Modified Example

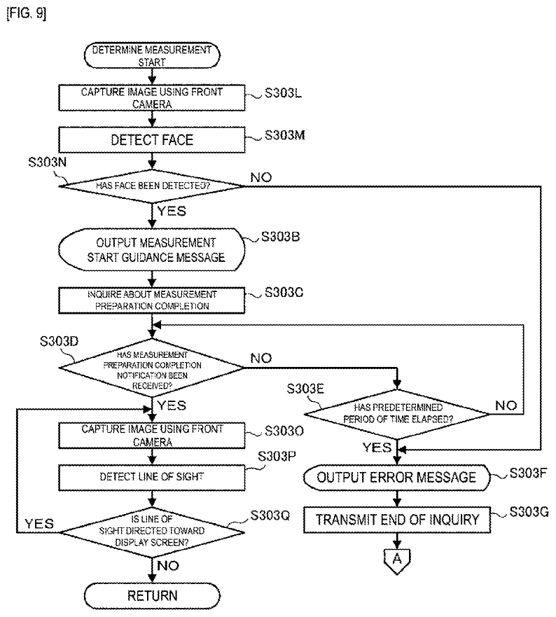

FIG. 9 is a flowchart illustrating an example of a processing procedure of measurement start determination processing in a second modified example of the first embodiment.

### [Step S303L]

In the second modified example, when the measurement start determination processing is started, the CPU 322 first captures an image using the front camera 311, and stores the captured image in the RAM 323 or the storage unit 302 via the image processing circuit 303.

### [Step S303M]

Then, the CPU 322 performs known face detection processing on the image from the front camera 311 stored in the RAM 323 or the storage unit 302. The program for the face detection processing may be stored in the storage unit 302 in advance as a part of the blood pressure measurement application program, or another application program may be utilized that is stored in the storage unit 302 for auto focus processing of the camera or face image processing, for example.

### [Step S303N]

Then, the CPU 322 determines whether a face is detected in the image from the front camera 311 through the face detection processing in step S303M described above. In a case where a face is determined to have been detected (step S303N, YES), the CPU 322 advances the processing to step S303B described above. In a case where no face is determined to have been detected (step S303N, NO), the CPU 322 advances the processing to step S303F described above.

### [Steps S303B to 5303G]

Steps S303B to 5303G are as described above in the first embodiment. However, in a case where in step S303N, no face is determined to have been detected (step S303N, NO) and where the processing proceeds to step S303F, transmission of the end of the inquiry in step S303G described above may be skipped.

### [Step S3030]

In step S303D described above, in a case where the measurement preparation completion notification is determined to have been received from the blood pressure monitor 200 (step S303D, YES), the CPU 322 captures an image again using the front camera 311. The image from the front camera 311 is stored in the RAM 323 or storage unit 302 via the image processing circuit 303.

### [Step S303P]

Then, the CPU 322 performs known line-of-sight detection processing on the image from the front camera 311 stored in the RAM 323 or storage unit 302. The program for the line-of-sight detection processing may be stored in the storage unit 302 in advance as a part of the blood pressure measurement application program, or another application program may be utilized that is stored in the storage unit 302 for character movement or character capturing in a game application program or key selection for a software keyboard based on the line of sight, for example.

### [Step S303Q]

Then, the CPU 322 determines, through the face detection processing in step S303P, whether, in the image from the front camera 311, the line of sight is directed toward the display screen 316. Here, the position of the front camera 311 with respect to the display screen 316 is stored in advance in the ROM 324 or the storage unit 302. Thus, the CPU 322 can easily determine whether the line of sight detected is directed toward the display screen 316. In a case where the line of sight is determined to be directed toward the display screen 316 (step S303Q, YES), the CPU 322 returns the processing to step S3030. In a case where the line of sight is determined to be away from the display screen 316 (step S303Q, NO), the CPU 322 determines that the measurement start condition is satisfied, ends the measurement start determination processing, and advances the processing to step S304. Thus, the CPU 322 can execute the processing of the determination unit 41 in which the measurement start condition is determined to be satisfied when the line of sight of the operator is away from the display screen 38, as described above in the application example.

In this way, the measurement start condition may be that the line of sight of the operator corresponding to the subject is away from the display screen 316. When the operator performs the start operation, the operator views the display screen 316. Thus, for example, in a case where the line of sight of the operator is detected by a known line-of-sight detection technique utilizing the front camera 311 and where the line of sight detected is away from the display screen, the operator can be determined to be prevented from viewing the display content of the display screen 316. Thus, in the second modified example, the measurement start condition is that the line of sight of the operator is away from the display screen 316. This allows the subject to be easily determined to be prevented from viewing the display content of the display screen 316.

### <1-4-3> Third Modified Example

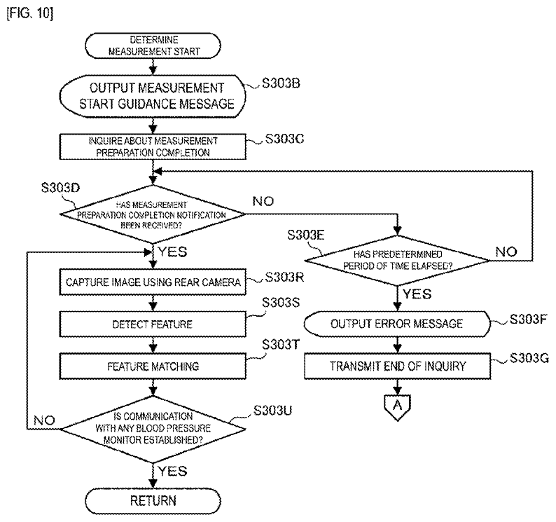

FIG. 10 is a flowchart illustrating an example of a processing procedure of measurement start determination processing in a third modified example of the first embodiment.

### [Steps S303B to S303G]

In the third modified example, when the measurement start determination processing is started, the CPU 322 performs the processing in steps S303B to S303G described above without acquiring the initial angle of the display screen as in step S303A of the first embodiment.

### [Step S303R]

In step S303D described above, in a case where the measurement preparation completion notification is determined to have been received from the blood pressure monitor 200 (step S303D, YES), the CPU 322 captures an image using the rear camera 312. The image from the rear camera 312 is stored in the RAM 323 or the storage unit 302 via the image processing circuit 303.

### [Step S303S]

Then, the CPU 322 performs known feature detection processing on the image from the rear camera 312 stored in the RAM 323 or the storage unit 302. The feature detection processing is, for example, processing in which edge detection or the like is performed on the image and in which the shapes of objects included in the image, characters, and the like are detected. The program for the feature detection processing may be stored in the storage unit 302 in advance as a part of the blood pressure measurement application program, or another application program may be utilized that is stored in the storage unit 302. The features detected are stored in the RAM 323 or the storage unit 302.

### [Step S303T]

Subsequently, the CPU 322 performs feature matching processing. This processing matches features stored in the RAM 323 or the storage unit 302 with feature information such as the shape of the blood pressure monitor 200 stored in advance in the storage unit 302 as a part of the blood pressure measurement application program, the logo of the product name, a model number string, and the like. For example, the storage unit 302 stores feature information regarding a plurality of types of blood pressure monitors 200. The information may be optionally downloadable by the wireless communication interface 309 from an external device such as a server device via the communication network NW (Internet). When actual matching is performed, the processing time may be shortened by matching the features only with feature information corresponding to the blood pressure monitor 200 with which communication is established in step S301, from among feature information regarding the plurality of types of blood pressure monitors 200.

### [Step S303U]

Then, based on the results of the feature matching processing in step S303T described above, the CPU 322 determines whether the image from the rear camera 312 contains an image of the blood pressure monitor 200 with which communication is established in step S301. In a case where no image of the blood pressure monitor 200 with which communication is established is determined to be present (step S303T, NO), the CPU 322 returns the processing to step S303R described above. In a case where an image of the blood pressure monitor 200 with which communication is established is determined to be present (step S303T, YES), the CPU 322 ends the measurement start determination processing, and advances the processing to step S304 described above. Thus, when the operation unit 33 receives the start operation, the CPU 322 can perform the processing of the determination unit 41 in which the measurement start condition is determined to be satisfied in a case where the blood pressure monitor 2 is present opposite to the display screen 38 with respect to the information processing device 3, as described above in the application example.

In this way, the measurement start condition may be that the blood pressure monitor 200 is present at the rear surface, which is opposite to the display screen 316. In a case where the operator operating the information processing device 300 differs from the subject of the blood pressure, the positional relationship between the subject and the information processing device 300 varies, and it is difficult to determine which orientation of the display screen 316 prevents the subject from viewing the display content of the display screen 316. However, at least in a case where the blood pressure monitor 200 is present on the rear surface side of the information processing device 300 when the start operation is performed, the subject with the blood pressure monitor 200 is reliably located opposite to the display screen 316, that is, the subject is prevented from viewing the display screen 316. Thus, in the third modified example, the rear camera 312 is utilized to confirm the presence of the blood pressure monitor 200, and the measurement start condition is that the blood pressure monitor 200 is present. This enables easy determination that the subject is prevented from viewing the display content of the display screen 316.

### <1-4-4> Fourth Modified Example

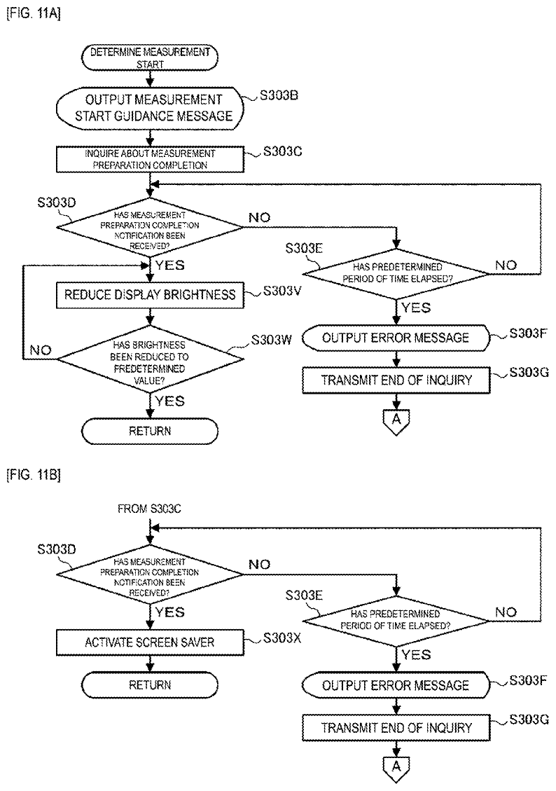

FIG. 11A is a flowchart illustrating an example of a processing procedure of measurement start determination processing in a fourth modified example of the first embodiment.

### [Steps S303B to S303G]

In the fourth modified example, when the measurement start determination processing is started, the CPU 322 performs the processing in steps S303B to S303G described above without acquiring the initial angle of the display screen as in step S303A of the first embodiment.

### [Step S303V]

In step S303D described above, in a case where the measurement preparation completion notification is determined to have been received from the blood pressure monitor 200 (step S303D, YES), the CPU 322 causes the graphics controller 306 to reduce the brightness (luminance) of the display screen 316.

### [Step S303W]

Then, the CPU 322 determines whether the brightness (luminance) of the display screen 316 has been reduced to a predetermined brightness (luminance). The predetermined brightness (luminance) is set to a value at which the display content of the display screen 316 is invisible. For example, the predetermined brightness (luminance) is stored in the storage unit 302 in advance as a part of the blood pressure measurement application program. In a case where it is determined that the display screen 316 has not been reduced to a predetermined brightness (luminance) (step S303W, NO), the CPU 322 returns the processing to step S303V above. In a case wherein the brightness (luminance) of the display screen 316 is determined to have been reduced to a predetermined brightness (luminance) (step S303W, YES), the CPU 322 determines that the measurement start condition is satisfied, ends the measurement start determination processing, and advances the processing to step S304. Thus, the CPU 322 can execute the processing of the determination unit 41 in which, when the display screen 38 is dimmed enough to make the display content of the display screen 38 invisible, the measurement start condition is determined to be satisfied, as described above in the application example.

In this way, when the start operation is received, the display on the display screen 316 is gradually dimmed, and the measurement start condition may be that the display is dimmed enough to be invisible. Forcibly dimming the display screen 316 prevents the subject from viewing the display content of the display screen 316. However, sudden erasure of the display screen may be mistaken for a failure or may surprise the subject, adversely affecting the blood pressure measurement. Thus, in the fourth modified example, the display on the display screen 316 is gradually dimmed. In a case where the measurement start condition is that the display screen 316 is dimmed enough to make the display content of the display screen 316 invisible, the subject can be easily determined to be prevented from viewing the display content of the display screen 316.

FIG. 11B is a flowchart illustrating another example of a processing procedure of measurement start determination processing in the fourth modified example of the first embodiment.

### [Steps S303B to S303G]

When the measurement start determination processing is started, the CPU 322 performs the processing in steps S303B to S303G described above without acquiring the initial angle of the display screen as in step S303A of the first embodiment.

### [Step S303X]

In step S303D described above, in a case where the measurement preparation completion notification is determined to have been received from the blood pressure monitor 200 (step S303D, YES), the CPU 322 causes the graphics controller 306 to activate any screen saver to display the screen saver on the display screen 316. When the screen saver is displayed, the CPU 322 determines that the measurement start condition is satisfied, ends the measurement start determination processing, and advances the processing to step S304. Thus, the CPU 322 can perform the processing of the determination unit 41 in which, when the display on the display screen 38 changes to the state in which the subject is prevented from viewing the display content, the measurement start condition is determined to be satisfied, as described above in the application example.

In this way, when the start operation is received, the display on the display screen 316 may be switched to the screen saver. Displaying the screen saver on the display screen 316 ensures that the subject is prevented from viewing the display content of the display screen 316. In this way, in a case where the measurement start condition is that the display on the display screen 316 changes to the state of a screen saver state in which display content of the display screen 316 is invisible, the subject can be easily determined to be prevented from viewing the display content of the display screen 316.

### <2> Second Embodiment

Now, a second embodiment will be described.

### <2-1> Configuration and <2-2> Operation

A basic configuration and a basic operation of the biometric data measurement system according to the second embodiment are similar to the basic configuration and basic operation of the biometric data measurement system according to the first embodiment described above. Thus, descriptions of the matters described in the first embodiment are omitted, and items that can be easily inferred from the first embodiment described above are also omitted.

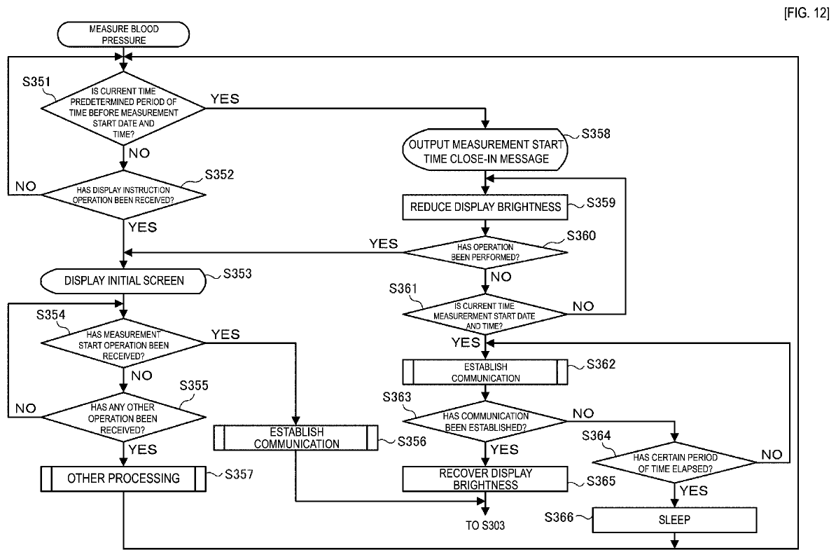

The present embodiment differs from the first embodiment described above in the processing procedure for the information processing device 300. FIG. 12 is a flowchart illustrating an example of a processing procedure for the information processing device 300 in accordance with a blood pressure measurement application program in the biometric data measurement system according to the second embodiment. For example, when the information processing device 300 is activated, the blood pressure measurement application program is executed in the background to start the processing procedure.

### [Step S351]

First, the CPU 322 of the information processing device 300 determines whether the current time clocked by a clock circuit 310 is a predetermined period of time before the measurement start date and time indicated by a measurement schedule stored in the storage unit 302. The predetermined period of time is set to the time required for the subject to complete preparation of blood pressure measurement such as attachment of the cuff 202 of the blood pressure monitor 200; the set time is, for example, 3 minutes. The 3 minutes are illustrative and no such limitation is intended. For example, the predetermined period of time is stored in the storage unit 302 in advance as a part of the blood pressure measurement application program. In a case where the current time is determined not to be the predetermined period of time before the measurement start date and time or the measurement start date and time is determined not to be set (step S351, NO), the CPU 322 advances the processing to step S352. In a case where the current time is determined to be the predetermined period of time before the measurement start date and time (step S351, YES), the CPU 322 advances the processing to step S358.

### [Step S352]

In step S351 described above, in a case where the current time is determined not to be the predetermined period of time before the measurement start date and time or the measurement start date and time is determined not to be set (step S351, NO), the CPU 322 determines whether the operator has performed a display instruction operation for an initial screen of the blood pressure measurement application program. The CPU 322 determines, for example, whether an activation icon for the blood pressure measurement application program on the display screen 316 has been operated or a specified operation of the running blood pressure measurement application program has been performed in a predetermined area such as a notification area in the display screen 316, for example, based on coordinate data provided by the touch panel controller 307 in response to a touch operation of the touch panel 317. In a case where the initial screen display instruction operation is determined not to have been performed (step S352, NO), the CPU 322 returns the processing to step S351 described above. In a case where the initial screen display instruction operation is determined to have been performed (step S352, YES), the CPU 322 advances the processing to step S353.

### [Step S353]

In step S352 described above, in a case where the initial screen display instruction operation is determined to have been performed (step S352, YES), the CPU 322 displays, on the display screen 316 via the graphics controller 306, the initial screen of the blood pressure measurement application program including the icon 38A for instructing the start of the blood pressure measurement.

### [Step S354]

Subsequently, the CPU 322 determines whether the start operation for the blood pressure measurement has been performed by the operator. For example, the CPU 322 determines, based on touch coordinates input from touch panel controller 307, whether the location on the touch panel 317 corresponding to the icon 38A for instructing the start of blood pressure measurement of touch panel 317 has been touched. In a case where the blood pressure measurement start operation is determined not to have been performed (step S354, NO), the CPU 322 advances the processing to step S355. In a case where the blood pressure measurement start operation is determined to have been performed (step S354, YES), the CPU 322 advances the processing to step S356.

### [Step S355]

In step S354 described above, in a case where the blood pressure measurement start operation is determined not to have been performed (step S354, NO), the CPU 322 determines whether any other operation has been performed in the initial screen. In a case where no other operation is determined to have been performed (step S355, NO), the CPU 322 returns the processing to step S354 described above. That is, in steps S354 and S355, the CPU 322 waits for an operation from the operator. In a case where any other operation is determined to have been performed (step S355, YES), the CPU 322 advances the processing to step S357.

### [Step S356]

In step S354 described above, in a case where the blood pressure measurement start operation is determined to have been performed (step S354, YES), the CPU 322 establishes near field communication with the blood pressure monitor 200 through the communication interface 305. Subsequently, the CPU 322 advances the processing to step S303 as described in the first embodiment.

### [Step S357]

In step S355 described above, in a case where any other operation is determined to have been performed (step S355, YES), the CPU 322 executes other processing in accordance with the operation. Examples of the other processing may include, for example, setting of a measurement schedule such as measurement of the blood pressure at 7 o'clock every morning, instruction to end display of the initial screen, instruction to end or prohibit of background execution of the blood pressure measurement application program, and the like. After the other processing ends, the CPU 322 returns the processing to step S351 described above.

### [Step S358]

On the other hand, in step S351 described above, in a case where the current time is the predetermined measurement start time before the measurement start date and time (step S351, YES), the CPU 322 outputs a measurement start time close-in message through the display screen 316 and/or the speaker 314. For example, the CPU 322 causes the graphics controller 306 to display the characters of a measurement start time close-in message on the display screen 316 and causes the sound processing unit 304 to emit the voice of a measurement start time close-in message through the speaker 314. For example, the content of the measurement start time close-in message is stored in the storage unit 302 in advance as a part of the blood pressure measurement application program. Note that even in a case where the information processing device 300 is in a sleep state, that is, even in a case where the operator is not using the information processing device 300, the measurement start time close-in message is output to notify the operator corresponding to the subject that the blood pressure measurement date and time closes in. Thus, the CPU 322 can perform the processing of the notification unit 43 as described above in the application example.

### [Step S359]

Subsequently, the CPU 322 causes the graphics controller 306 to reduce the brightness (luminance) of the display screen 316.

### [Step S360]

Then, the CPU 322 determines whether any operation has been performed by the operator. In a case where any operation is determined to have been performed (step S360, YES), the CPU 322 advances the processing to step S353 described above. In a case where no operation is determined to have been performed (step S360, NO), the CPU 322 advances the processing to step S361.

### [Step S361]

In step S360 described above, in a case where no operation is determined to have been operated (step S360, NO), the CPU 322 determines whether or not the current time clocked by the clock circuit 310 is past the measurement start date and time indicated by the measurement schedule stored in the storage unit 302. In a case where the current time is determined to be past the measurement start date and time (step S361, YES), the CPU 322 advances the processing to step S362. In a case where the current time is determined not to be past the measurement start date and time (step S361, NO), the CPU 322 returns the processing to step S359 described above. In this way, the display on the display screen 316 is gradually dimmed

Even in a case where the operator is executing an application program other than the blood pressure measurement application program, the processing in steps S359 to S361 inhibits the display and operation of this application program and divert the subject to the blood pressure measurement. However, in a case where the display is dimmed enough to make the display content completely unrecognizable, the operator may misinterpret the information processing device 300 as being in the sleep state. Thus, desirably, the display is dimmed such that the display content is recognizable to some degree to remind the operator that the blood pressure measurement date and time closes in. Then, when the operator performs some operation, regardless of the content of the operation, the processing can be shifted to step S353 described above to display the initial screen of the blood pressure measurement application program.

### [Step S362]

In step S361 described above, in a case where the current time is determined to be or be past the measurement start date and time (step S361, YES), the CPU 322 performs processing for establishing near field communication with the blood pressure monitor 200 through the communication interface 305.

### [Step S363]

Subsequently, the CPU 322 determines whether communication with the blood pressure monitor 200 has successfully been established. In a case where a long time is required for the subject corresponding to the operator to power on the blood pressure monitor 200, the communication establishment processing in step S362 ends with no communication established. In a case where communication with the blood pressure monitor 200 is determined to have successfully been established (step S363, YES), the CPU 322 advances the processing to step S365. In a case where communication is determined not to have successfully been established (step S363, NO), the CPU 322 advances the processing to step S364.

### [Step S364]

In step S363 described above, in a case where communication with the blood pressure monitor 200 is determined not to have successfully been established (step S363, NO), the CPU 322 determines whether a certain amount of time has elapsed. The certain amount of time is set shorter than the predetermined period of time in step S351, and set to, for example, 1 minute. The 1 minute is illustrative and not such limitation is intended. For example, the certain amount of time is stored in the storage unit 302 in advance as a part of the blood pressure measurement application program. In a case where the certain amount of time is determined not to have elapsed (step S364, NO), the CPU 322 returns the processing to step S362 described above. In a case where the certain amount of time is determined to have elapsed (step S364, YES), the CPU 322 advances the processing to step S366.

### [Step S365]

In step S363 described above, communication with blood pressure monitor 200 is determined to have successfully been established (step S363, YES), the CPU 322 causes the graphics controller 306 to recover the brightness (luminance) of display screen 316 to the original brightness. Subsequently, the CPU 322 advances the processing to step S303 as described in the first embodiment. Specifically, establishment of communication with the blood pressure monitor 200 means that the subject has prepared for the blood pressure measurement. Thus, in this case, whether the measurement start operation has been performed or not is not determined, and processing is executed that is similar to the processing performed in a case where the measurement start operation has been executed.

### [Step S366]

In step S364 described above, in a case where a certain period of time is determined to have elapsed with no communication with the blood pressure monitor 200 established (step S364, YES), the CPU 322 transitions the information processing device 300 into the sleep state. The CPU 322 then returns the processing to step S351 described above.

### <2-3> Effect

According to the second embodiment described above, in addition to the blood pressure measurement at any point in time instructed by the operator as in the first embodiment described above, blood pressure measurement according to the measurement schedule specified by the subject in advance may be performed.

In other words, in a case where a preset date and time closes in, for example, the CPU 322 outputs a measurement start time close-in message, and then gradually dims the display on the display screen 316. Then, in a case where communication with the blood pressure monitor 200 is established within a certain period of time from the measurement start date and time, the display is recovered to the original state, and a determination of whether the measurement start condition is satisfied is started. In this way, notifying the subject that the measurement start date closes in can prompt the subject to measure the blood pressure at the preset date and time, and prevent the subject from forgetting to measure the blood pressure. Moreover, the display is gradually dimmed after the measurement start time close-in message is output, and thus even in a case where the subject is operating any other application program on the information processing device 300, the operation is made difficult, allowing the subject to be diverted to the blood pressure measurement.

Note that, rather than outputting the measurement start time close-in message when the preset date and time closes in, a measurement start time arrival message may be output when the current time is or is past the preset date and time. In other words, instead of using, as a trigger, the time the predetermined period of time before the measurement start date, the measurement start date and time may be used as a trigger. In this case, the determination in step S361 may be omitted.

Note that the embodiments have been described but that the present invention is not limited to the embodiments described above, and can be embodied by modifying the components in an implementation stage without departing from the gist thereof. Further, various inventions can be formed by appropriately combining a plurality of constituent elements disclosed in the embodiment described above. For example, some constituent elements may be omitted from the entire constituent elements shown in the embodiment. Furthermore, the constituent elements of different embodiments may be combined appropriately.

For example, the embodiments have been described taking, as an example, an electronic blood pressure monitor using a technique (oscillometric method) for compressing the blood vessel to detect a pulse wave. However, the present invention can be applied to, for example, blood pressure monitors using any techniques, such as a technique for continuously measuring the blood pressure of a measurer based on a pulse transit time (PTT), a technique for continuously measuring the blood pressure based on a pressure pulse wave (tonometry method), and a technique for detecting a pulse wave using a light emitting element. In a blood pressure monitor that does not use the oscillometric method, the measurement may be immediately discontinued, for example, when an emergency stop instruction is input or when a measurement stop command is received from the information processing device. The inquiry of whether the measurement preparation is complete can be omitted, the inquiry being made to the blood pressure monitor from the information processing device.

The whole or part of the embodiments described above can be described as, but not limited to, the following supplementary notes.

### (Supplementary Note 1)

A method for causing a biometric data measurement device to measure biometric data, the method being executed by an information processing device including a processor, a memory, a communication unit connecting to a communication network, a display unit including a display screen displaying various pieces of information, a connection unit connecting to another device wirelessly or by wire, and an operation unit receiving a user operation, the method including the steps of:

the processor determining whether a measurement start condition is satisfied when the operation unit receives a measurement start operation for the biometric data measuring device, and
the processor transmitting a measurement start command to the biometric data measurement device via the connection unit in a case where the measurement start condition is satisfied,
wherein the measurement start condition includes prevention of the subject of the biometric data from viewing a display content of the display screen.

### (Supplementary Note 2)

The biometric data measurement system according to claim 1 or 2, wherein the information processing apparatus further includes a speaker generating sound, and the control unit controls to output, through the speaker, music assisting in maintaining a resting state of the subject during measurement performed by the biometric data measurement system.

### (Supplementary Note 3)

The biometric data measurement system according to claim 2, wherein the measurement start angle is an angle exceeding a range of ±90 degrees with respect to the angle of the display screen when the operation unit receives the start operation.

### (Supplementary Note 4)

The biometric data measurement system according to claim 10, wherein, when the control unit gradually dims display on the display screen, in a case where the control unit is connected to the biometric data measurement device within a certain period of time, the control unit recovers the display on the display screen to an original state and then starts to determine whether the measurement start condition is satisfied.

### (Supplementary Note 5)

The biometric data measurement system according to any one of claims 1 to 14, wherein the control unit erases the display screen when the measurement start condition is satisfied.

### (Supplementary Note 6)

A biometric data measurement program causing a processor to function as the control unit included in the information processing device according to any one of claims 1 to 15.

### REFERENCE SIGNS LIST

1 Biometric data measurement system
2, 200 Blood pressure monitor
3, 300 Information processing device
21, 201 Blood pressure monitor body
22, 202 Cuff
23 Blood pressure measurement unit
24, 203 Fluid bag
25 Detection unit
26 Blood pressure monitor connection unit
27 Blood pressure monitor control unit
28 Emergency stop button
31 Communication unit
32 Connection unit
33 Operation unit
33A Operation button
33B Touch sensor
34, 315 Display unit
35 Sensor
35A, 311 Front camera
35B, 319 Illuminance sensor
36 Notification unit
37, 204, 301 Control unit
38, 316 Display screen
38A Icon
39, 314 Speaker
40 Vibration member
41 Determination unit
42 Operation guidance unit
43 Notification unit
44 Inquiry unit
205 Oscillation circuit
206 Pump drive circuit
207 Valve drive circuit
208, 308 Input/output interface
209, 305 Communication interface
210 Pressure sensor
211 Piezoelectric pump
212 Valve
213 Tubing
214 Cuff wrapping state detection unit
215 Power supply switch
216, 322 CPU
217, 323 RAM
218, 324 ROM
302 Storage unit
303 Image processing circuit
304 Sound processing unit
306 Graphics controller
307 Touch panel controller
309 Wireless communication interface
310 Clock circuit
312 Rear camera
313 Microphone
317 Touch panel
318 Acceleration sensor
320 Operation member
321 Vibrator

1. A biometric data measurement system comprising:
a biometric data measurement device configured to measure biometric data of a subject; and
an information processing device configured to control a biometric data measurement operation by the biometric data measurement device, wherein
the information processing device includes:
a communication unit configured to connect to a communication network;
a display unit including a display screen displaying various pieces of information;
a connection unit configured to connect to the biometric data measurement device wirelessly or by wire;
an operation unit configured to receive a start operation for a measurement operation for biometric data of the subject by the biometric data measurement device; and
a control unit configured to transmit a measurement start command for starting measurement of the biometric data, to the biometric data measurement device via the connection unit when a measurement start condition is satisfied after the operation section receives the start operation, and
the measurement start condition includes prevention of the subject from viewing a display content of the display screen.

2. The biometric data measurement system according to claim 1, wherein
the information processing device further includes
a sensor configured to detect an angle of the display screen with respect to a gravitational direction, and
the control unit includes
a determination unit configured to determine that the measurement start condition is satisfied when the angle of the display screen equals a measurement start angle at which the subject is prevented from viewing the display screen.

3. The biometric data measurement system according to claim 1, wherein
the information processing device further includes
a sensor configured to detect that the display screen is covered, and
the control unit includes
a determination unit configured to determine that the measurement start condition is satisfied when the display screen is covered.

4. The biometric data measurement system according to claim 1, wherein
the information processing device further includes
a sensor configured to detect a line of sight of an operator operating the operation unit, and
the control unit includes
a determination unit configured to determine that the measurement start condition is satisfied when the line of sight of the operator is away from the display screen.

5. The biometric data measurement system according to claim 1, wherein
the information processing device further includes
a sensor configured to detect that the biometric data measurement device is present opposite to the display screen with respect to the information processing device, and
the information processing device includes
a determination unit configured to determine, when the operation unit receives the start operation, that the measurement start condition is satisfied in a case where the biometric data measurement device is present opposite to the display screen with respect to the information processing device.

6. The biometric data measurement system according to claim 1, wherein
the control unit controls the display unit such that when the operation unit receives the start operation, the display unit changes display on the display screen to a display state in which the subject is prevented from viewing the display, and
the control unit includes
a determination unit configured to determine that the measurement start condition is satisfied when the display content of the display screen changes to the display state in which the subject is prevented from viewing the display content.

7. The biometric data measurement system according to claim 1, wherein
the information processing device further includes
a notification unit including at least one of a speaker configured to generate sound and a vibration member configured to generate vibration, and
the control unit causes, after elapse of a certain period of time since satisfaction of the measurement start condition, the notification unit to notify the subject of start of measurement, and
the control unit transmits, after notification of the start of measurement by the notification unit, the measurement start command to the biometric data measurement device via the connection unit.

8. The biometric data measurement system according to claim 7, wherein
the control unit prohibits operation of the notification unit during measurement of the biometric data by the biometric data measurement device, and
when a measurement result for the biometric data is received from the biometric data measurement device via the connection unit, the control unit causes the notification unit to notify the subject of end of the measurement.

9. The biometric data measurement system according to claim 1, wherein
the control unit includes
an operation guidance unit configured to display a guidance message prompting movement of the information processing device to satisfy the measurement start condition, on the display screen when the operation unit receives the start operation.

10. The biometric data measurement system according to claim 1, wherein
the control unit includes
a notification unit configured to display a message promoting biometric data measurement with the biometric data measurement device, on the display screen at a preset date and time, and
the control unit gradually dims the display on the display screen after the notification unit outputs the message.

11. The biometric data measurement system according to claim 1, wherein
the biometric data measurement device includes a blood pressure monitor that includes
a blood pressure measurement unit including a cuff containing a fluid bag and wrapped around a measured site of the subject and configured to measure a blood pressure of the subject;
a detection unit configured to detect a state of wrapping, around the measured site, of the cuff of the blood pressure measurement unit;
a blood pressure monitor connection unit connected wirelessly or by wire to the information processing device, and
a blood pressure monitor control unit configured to cause the blood pressure measurement unit to measure the blood pressure of the subject when the measurement start command is received from the information processing device via the blood pressure monitor connection unit, and transmit a result of the measurement to the information processing device via the blood pressure monitor connection unit.

12. The biometric data measurement system according to claim 11, wherein
the control unit includes
an inquiry unit configured to inquire of the blood pressure monitor, via the connection unit, whether or not preparation for measurement with the blood pressure measurement unit is completed, and
when the inquiry is received from the information processing device, the blood pressure monitor control unit transmits a measurement preparation completion notification to the information processing device via the blood pressure monitor connection unit in a case where a state of wrapping of the cuff around the measured site detected by the detection unit corresponds to a blood pressure measurement enabled state, and
when the measurement preparation completion notification is received, the control unit starts determining whether the measurement start condition is satisfied.

13. The biometric data measurement system according to claim 11, wherein
when the measurement start condition is no longer satisfied after the measurement start command is transmitted to the blood pressure monitor, the control unit transmits a measurement stop command to the blood pressure monitor via the connection unit, and
when the blood pressure monitor control unit receives the measurement stop command from the information processing device, in a case where a compressed state by the fluid bag contained in the cuff of the blood pressure measurement unit continues for a certain period of time or longer, the blood pressure monitor control unit discontinues the measurement of the blood pressure and causes a fluid in the fluid bag to be discharged.

14. The biometric data measurement system according to claim 11, wherein
the blood pressure monitor further includes
an emergency stop button dedicated to emergency stop or also used as another button, and
in response to an operation of the emergency stop button, the blood pressure monitor control unit discontinues the measurement of the blood pressure and cause the fluid in the fluid bag contained in the cuff of the blood pressure measurement unit to be discharged.

15. An information processing device comprising:
a communication unit configured to connect to a communication network;
a display unit including a display screen displaying various pieces of information;
a connection unit configured to connect to another device wirelessly or by wire;
an operation unit configured to receive a user operation; and
a control unit configured to control each of the units, wherein
the control unit
determines whether a measurement start condition is satisfied when the operation unit receives a measurement start operation for a biometric data measuring device measuring biometric data of a subject, and
transmits a measurement start command to the biometric data measurement device via the connection unit in a case where the measurement start condition is satisfied, and
the measurement start condition includes prevention of the subject from viewing a display content of the display screen.
